(19) United States
(12) Patent Application Publication
(10) Pub. No.: US 2019/0262188 A1
(43) Pub. Date: Aug. 29, 2019

(54) Tension adjusting and stabilization system and method

(71) Applicant: Lawrence G. Reid, JR., Germanton, NC (US)

(72) Inventor: Lawrence G. Reid, JR., Germanton, NC (US)

(21) Appl. No.: 16/350,597

(22) Filed: Dec. 7, 2018

Related U.S. Application Data
(63) Continuation-in-part of application No. 14/225,952, filed on Mar. 26, 2014, now abandoned.

(51) Int. Cl. A61F 13/08; D04B 1/18

(52) U.S. Cl. CPC A61F 13/085; A61F 2013/00119; D04B 1/18

(57) ABSTRACT

A tension adjusting and stabilization system and method can include an elastic compression bandage including long-stretch elastomeric yarns and an approximate 1:1 stretch characteristic. A change in length-wise stretch of the bandage results in an approximately proportional change in compressive pressure provided by the bandage. The system and method can further include a means for indicating a qualitative degree of tension of the bandage and a means for indicating a quantitative amount of pressure being applied by that degree of tension.

### TECHNICAL FIELD

The subject matter described herein relates to a compression and sensing system and method, which can include a sleeve-wrap compression system and method and a body monitoring system and method, and to a tension adjusting and stabilization system and method.

### BACKGROUND

Compressive pressure is utilized in the treatment and/or prevention of wounds, peripheral vascular disease, leg ulcers, edema, lymphatic disorders, and other conditions. Compressive pressure can be applied by compression garments, wraps, and/or bandages (collectively referred to as compression devices). In many conventional compression device applications, the actual amount of compressive force provided by the device at its interface with an anatomical area when worn is unknown. To provide effective clinical management of compression therapy, the amount of compressive pressure being applied with a particular device to a patient needs to be accurate. Insufficient compression may result in suboptimal treatment. Excessive compression can retard blood flow, leading to detrimental results.

The need for accurate application of compressive pressure is further driven by the fact that clinical needs between patients often vary. For example, an early stage leg ulcer may need a low level of compression, while a severe case of lymphedema may require higher compression levels. Accurate application of compressive pressure is also important to provide graduated pressure along an anatomical location, such as a leg.

Another clinical situation in which it is important to know the actual compressive pressure being applied is when the patient has a reduction in edema underneath the compression garment. If the reduction in edema is sufficient to affect the amount of compressive pressure being applied, a smaller compression garment, a garment that provides a greater amount of pressure (such as a more tightly wrapped bandage), or an additional compressive pressure layer may need to be applied. As an example, some compressive pressure systems apply compression with a high stiffness, or rigidity, factor. With a reduction in edema, a rigid compression system becomes unable to provide compression as the underlying anatomical area reduces in diameter and pulls away from the compression system. In this instance, knowing the actual compressive pressure being applied provides the information necessary to determine whether the compression system may need to be replaced in order for therapy to be continued.

An amount of compressive pressure that a garment is capable of providing when applied to a patient can be determined prior to use. Compression fabrics/garments can be tested under stretch conditions and certified for compression ranges within a defined circumference fitting range. The amount of compression that a fabric or garment is capable of generating can be affected by various yarn and construction factors. Such factors can include, for example, yarn type and size (for example, denier); characteristics of elastic yarns utilized (for example, how an elastic yarn is extruded and/or wrapped, such as under how much tension); and fabric structure (for example, stitch pattern, size, and/or density).

Applying accurate compressive pressure to a body with compression devices poses numerous challenges. The actual amount of compressive pressure applied by a particular device depends on various factors, including, for example, the number of fabric layers applied, the type and amount of elastic material in each layer, the combined stretch characteristics of multiple layers and/or materials, body shape and circumference, and other variables. For example, yarn fatigue (or yarn creep) can affect the ability of a device to provide compression. Yarn fatigue can be defined as the weakening of a yarn caused by a loss of some of its ability to recover to its original shape or size after being deformed repeatedly. As a result, a compression device over time can lose elasticity and the ability to provide the compressive force for which it was initially rated. Thus, it becomes important to determine the actual amount of compressive pressure the device provides after repeated and/or prolonged use.

Since applying a compressive wrap is somewhat subjective and can thus vary by user, how much tension and how much overlap to apply is another challenge to applying accurate compressive pressure to a body. Once applied, how well the device maintains a desired pressure when the circumference of an underlying body parts changes due to swelling and/or reduction of swelling can vary.

Another challenge to accurate application of compressive pressure relates to multi-layer compression systems. In conventional multi-layer bandaging, a combination of different types of bandage layers is used in order to provide an accumulation of pressure and to provide rigidity. Such bandages have disadvantages, including difficulty in applying the multiple layers of bandages to obtain a particular desired cumulative pressure and/or a relatively uniform pressure and to maintain that pressure over time. The application process can be time consuming. And, such bandages are prone to slipping and/or forming wrinkles after being applied, which may result in insufficient and/or uneven compression being applied, discomfort to the patient, and/or skin lesions. Thus, accurate application, and measurement, of actual applied compressive pressure is critical for proper use of multi-layer compression systems.

Determining actual applied interface compression on humans has proven difficult. Conventional compression devices that do attempt to provide measurement of actual applied compressive pressure are not accurate and are expensive. For example, current pneumatic pressure sensors used to record pressures developed beneath compression bandages or compression hosiery can exhibit reliability issues related to measurement sensitivity due to location of the air bladder on firm or soft tissue and variable sensitivity of the device itself.

Another disadvantage of such conventional compression devices is that they often have components that are reused from patient to patient, thereby increasing the risk of cross contamination, particularly when utilized in wound care.

Thus, there is a need for compression fabric, bandages, garments, and methods that allow accurate application to achieve desired pressures, that provide gradual changes in compression, and that allow compression to be maintained within a wide range of changes in circumference of underlying body parts.

There is a need for a means for easily and accurately determining an actual amount of interface compression applied at an anatomical area by a compressive pressure device. There is a need for such a means for easily and accurately determining an actual amount of applied compressive pressure that is reliable regardless of anatomical location tested and across repeated measurements. There is a need for such a means for easily and accurately determining an actual amount of applied compressive pressure the entire time the device/garment is being worn. There is a need for such a means for easily and accurately determining an actual amount of applied compressive pressure regardless of variables related to yarn, fabric construction, stretch characteristics, number of fabric layers, yarn/fabric fatigue, body shape and circumference, etc. There is a need for such a means for easily and accurately determining an actual amount of applied compressive pressure in a compression therapy system that reliably stays in place on a patient's limb and that maintains an initial working compression profile on the limb over time. In particular, there is a need for such a means for easily and accurately determining an actual amount of applied compressive pressure in a multi-layer compression therapy system. There is a need for such a means for easily and accurately determining an actual amount of applied compressive pressure that is economically constructed. There is a need for such a means for easily and accurately determining an actual amount of applied compressive pressure that decreases risk for cross contamination.

### SUMMARY

Some embodiments of the subject matter described herein include a tension adjusting and stabilization system and method. For example, some embodiments of a tension adjusting and stabilization system can include an elastic compression bandage comprising long-stretch elastomeric yarns and an approximate 1:1 stretch characteristic, wherein a change in length-wise stretch of the bandage results in an approximately proportional change in compressive pressure provided by the bandage. For example, each 10% increase in length-wise stretch of the bandage can produce an increase of 10 mm Hg compressive pressure provided by the bandage. In exemplary embodiments, the elastic compression bandage maintains pressure within about five percent of the initial applied pressure over at least a five-hour time period. In preferred embodiments, the elastomeric yarns comprise an about 420 denier spandex yarn.

In some embodiments, the tension adjusting and stabilization system can further comprise a repeating series of lines oriented transversely across at least a part of a width of the bandage at predetermined, spaced-apart intervals along a length of the bandage. Each line within the repeating series of lines can be labeled with an indicator representative of a particular amount of compressive pressure such that when the bandage is stretched about a limb and the lines having matching pressure indicators are aligned, the amount of compressive pressure indicated by the matching pressure indicators is provided.

In some embodiments, the elastic compression bandage can further comprise a pressure color indicator along a length of the bandage comprising one color in an unstretched condition that changes to a different color in a stretched condition, wherein the different, stretched color corresponds to a predetermined amount of stretch applied to the bandage, and wherein the predetermined amount of stretch applied to the bandage corresponds to a predetermined amount of compressive pressure. In such an embodiment, the pressure color indicator can further comprise a spaced-apart, repeating series of shapes along the length of the bandage, such that as the bandage is stretched, the shapes in a first one of the series are matched to the shapes in a second one of the series to provide a particular, known compressive pressure. A first shape in each series of shapes can comprise a first color, a second shape in each series can comprise a second color, and a third shape in each series can comprise a third color. As the bandage is stretched, the three colored shapes in a first one of the series can be matched to the colored shapes in another one of the series to provide a particular, known compressive pressure.

In some embodiments, the tension adjusting and stabilization system can further comprise a means for indicating a qualitative degree of tension of the bandage as it is being wrapped on a limb and a means for indicating a quantitative amount of pressure being applied by that degree of tension. The means for indicating a qualitative degree of tension can further comprise a vertical row of dark rectangular boxes printed onto the elastic compression bandage in an unstretched condition, and the means for indicating a quantitative amount of pressure can further comprise a mark representing a unique amount of pressure embedded in each of the dark rectangular boxes invisible in the unstretched condition. When sufficient tension is applied on the bandage to achieve the pressure invisible within one of the rectangular boxes, that rectangular box becomes a square shape and the unique amount of compressive pressure embedded in that rectangular box becomes visible.

In some embodiments, the tension adjusting and stabilization system can further comprise a tension pressure indicator printed in one or more locations on the bandage and comprising a rectangle shape having an oval shape inside the rectangle shape when the bandage is unstretched, wherein when the bandage is wrapped to a stretched position such that the rectangle shape becomes a square shape and the oval shape becomes a circle shape, the bandage provides a predetermined amount of compressive pressure.

Some embodiments of a tension adjusting and stabilization method can include providing an elastic compression bandage comprising long-stretch elastomeric yarns and an approximate 1:1 stretch characteristic, wherein a change in length-wise stretch of the bandage results in an approximately proportional change in compressive pressure provided by the bandage; and wrapping the bandage with a particular amount of tension at a certain level on a limb, thereby providing a known amount of pressure at that level of the limb. Such a method can further comprise providing two of the elastic compression bandages, each further comprising a tension pressure indicator printed in one or more locations on the bandage and comprising a rectangle shape having an oval shape inside the rectangle shape when the bandage is unstretched, wherein when the bandage is wrapped to a stretched position such that the rectangle shape becomes a square shape and the oval shape becomes a circle shape, the bandage provides a predetermined amount of compressive pressure. Such a method can further comprise wrapping a first of the two bandages to the stretched position such that the tension pressure indicator comprises the square and the circle; and wrapping a second of the two bandages with a 50% overlap of the first bandage such that the tension pressure indicator comprises the square and the circle, wherein each bandage provides the same amount of pressure, and the two bandages together provide a cumulative pressure approximately twice that of one bandage.

Some embodiments of a tension adjusting and stabilization system can include a wearable compressive pressure device comprising an elastic compression bandage comprising long-stretch elastomeric yarns and an approximate 1:1 stretch characteristic, wherein a change in length-wise stretch of the bandage results in an approximately proportional change in compressive pressure provided by the bandage; an electrically conductive yarn (a) comprising a 70 denier conductive yarn having 24-68 filaments, (b) knit vertically in a selected number of adjacent wales along a longitudinal axis of the device, (c) knit tightly such that stitch loops in adjacent courses along the wales are compacted together to enhance yarn and fiber contact, and (d) comprising a transmission circuit configured to transmit an electrical signal representing a compressive pressure value in an area of a body to a connection point on the transmission circuit; a sensor connectable to the transmission circuit and configured to sense compressive pressure in the area of a body to which the device is applied; and a data processor or display unit connectable to the transmission circuit and configured to display the transmitted compressive pressure value.

Some embodiments of the subject matter described herein include a compression and sensing system and method comprising a sleeve-wrap compression system and method. For example, some embodiments of a compression and sensing system and method can include a seamless, inner sleeve comprising a long-stretch elastomeric material and an interior terry surface; and an elongated outer wrap comprising a long-stretch elastomeric material. When applied to a patient's limb, the inner sleeve can exert a first compressive pressure that secures the inner sleeve in a therapeutic position on the limb. When applied by stretching over the inner sleeve, the outer wrap can exert a second compressive pressure and frictionally engage the inner sleeve, thereby securing the compression and sensing system as a single compressive entity in the therapeutic position on the limb.

In some embodiments, the first compressive pressure exerted by the inner sleeve can comprise about 5-10 mm Hg of compressive pressure uniformly throughout the sleeve. In some embodiments, the inner sleeve can further comprise a stitch construction that permits horizontal stretch with minimal vertical stretch. In some embodiments, the inner sleeve can further comprise a reciprocated heel pouch and an open toe, each adapted to guide application of the inner sleeve and to maintain the inner sleeve in the therapeutic position on the limb. In this way, wrinkling and/or bunching of the inner sleeve are reduced so that the inner sleeve compacts evenly onto the limb under the compressive pressure exerted by the outer wrap. The inner sleeve can be configured to disperse the compressive pressure exerted by the outer wrap smoothly about the therapeutic position on the limb.

In some embodiments, the second compressive pressure exerted by the outer wrap can comprise defined amounts of compressive pressure correlated with various amounts of stretch. In some embodiments, the outer wrap can further comprise a range of stretch to about 165% greater than a relaxed length. In some preferred embodiments, the second compressive pressure exerted by the outer wrap from a first stretch to an about 30% greater length than a relaxed length to a second stretch to an about 100% greater length than the relaxed length ranges from about 20 mm Hg to about 30 mm Hg of compressive pressure. For example, in some preferred embodiments, the outer wrap is configured to provide about 5-10 mm Hg compressive pressure when stretched to a first, about 30% greater length than a relaxed length, about 20 mm Hg compressive pressure when stretched to a second, about 75% greater length than the relaxed length, and about 30-35 mm Hg compressive pressure when stretched to a third, about 100% greater length than the relaxed length. In some embodiments, the outer wrap can further comprise a stitch construction that permits longitudinal stretch with minimal cross-stretch.

In some embodiments, the long-stretch elastomeric material in the outer wrap can comprise spandex having a denier of about 380-440. In some embodiments, the outer wrap can further comprise about 12-18 ends of spandex per inch.

In some embodiments, the first compressive pressure exerted by the inner sleeve and the second compressive pressure exerted by the outer wrap cumulatively comprise a working compression profile. In certain embodiments, the compression and sensing system further comprises an elastic stress/strain curve such that the single compressive entity provides a gradual change in the working compression profile in response to a change in limb volume. In certain other embodiments, the single compressive entity can maintain an initial working compression profile on the limb within a defined therapeutic range during changes in limb volume. In certain yet other embodiments, the single compressive entity can maintain an initial working compression profile on the limb with a variance of less than about 20% over a seven day period.

Embodiments of the compression and sensing system can further comprise a color/compression change indication system. In one embodiment of the color/compression change indication system, a particular amount of stretch of the outer wrap creates a unique shade of color representative of a particular amount of compressive pressure. In this way, a user can readily determine a proper amount of stretch for providing a desired amount of compressive pressure.

In some embodiments, each of the inner sleeve and the outer wrap further comprise broad spectrum anti-microbial properties. In some embodiments, each of the inner sleeve and the outer wrap further comprise a hydrophilic yarn adapted to wick moisture/fluid from a wound and surrounding skin to an outer surface of the outer wrap. For example, the inner sleeve hydrophilic yarn can comprise a knitted terry yarn.

In some embodiments, the compression and sensing system can further comprise a plurality of the outer wraps, wherein a second of the outer wraps can be applied on top of the first of the outer wraps in a three-layer system. In some embodiments, the outer wrap can comprise a cohesive wrap.

In some embodiments, the compression and sensing system can comprise a seamless sleeve comprising (a) a long-stretch elastomeric material, (b) a stitch construction that permits horizontal stretch with minimal vertical stretch, and (c) an interior terry surface. In such a system, when the sleeve is applied to a patient's limb, the sleeve exerts about 5-10 mm Hg of compressive pressure uniformly throughout the sleeve that secures the sleeve in a therapeutic position on the limb. In such an embodiment, the sleeve can be configured to have secured thereto a compression wrap overlying the sleeve. In some such embodiments, the sleeve can further comprise a reciprocated heel pouch and an open toe, each adapted to guide application of the sleeve and to maintain the sleeve in the therapeutic position on the limb. In such an embodiment, wrinkling and/or bunching of the sleeve are reduced and the sleeve compacts evenly onto the limb under compressive pressure exerted by the overlying compression wrap. The sleeve can also be configured to disperse the compressive pressure exerted by the overlying compression wrap smoothly about the therapeutic position on the limb.

In some embodiments, the compression and sensing system can comprise an elongated wrap comprising (a) a long-stretch elastomeric material, (b) a stitch construction having minimal cross-stretch, and (c) a range of longitudinal stretch to about 165% greater than a relaxed length. In such a system, when the wrap is applied to a patient's limb, the wrap exerts a compressive pressure that secures the wrap in a therapeutic position on the limb. In such a system, the compressive pressure exerted by the wrap can comprise defined amounts of compressive pressure correlated with various amounts of longitudinal stretch. In such a system, the compressive pressure exerted by the wrap from a first stretch to an about 30% greater length than the relaxed length to a second stretch to an about 100% greater length than the relaxed length can range from about 20 mm Hg to about 30 mm Hg of compressive pressure. For example, the wrap can be configured to provide about 5-10 mm Hg compressive pressure when stretched to a first, about 30% greater length than the relaxed length, about 20 mm Hg compressive pressure when stretched to a second, about 75% greater length than the relaxed length, and about 30-35 mm Hg compressive pressure when stretched to a third, about 100% greater length than the relaxed length. In some embodiments of such a system, the long-stretch elastomeric material in the wrap can comprise spandex having a denier of about 380-440, and the wrap can further comprise about 12-18 ends of spandex per inch.

Some embodiments of the subject matter described herein include a compression and sensing system and method comprising a body monitoring system and method. For example, some embodiments of a compression and sensing system and method can include a wearable device, and a circuit for conducting electrical signals comprising an electrically conductive yarn knitted into the device. In some embodiments, the circuit can further comprise a sensor circuit configured to sense a variable in an area of a body to which the device is applied. In some embodiments, the circuit can further comprise a transmission circuit configured to transmit an electrical signal representing a value of a variable in an area of a body to another location. The sensor circuit can further comprise an electrical sensitivity for reliably sensing the variable. The transmission circuit can further comprise an electrical sensitivity for reliably transmitting the value of a variable.

In some embodiments, the electrically conductive yarn can comprise a silver yarn or a yarn coated with silver. For example, the electrically conductive yarn can be a single 70 denier silver yarn or two 70 denier silver yarns twisted together. In embodiments in which the electrically conductive yarn comprises stitch loops, the stitch loops are preferably packed together during knitting so that the stitch loops in adjacent courses along a particular wale have sufficient contact to provide a continuous circuit. In embodiments in which the electrically conductive yarn comprises nylon yarn having silver or a silver composition applied thereto, the nylon yarn can be heated sufficiently to shrink the nylon yarn so that stitch loops in adjacent courses along a particular wale have sufficient contact to provide a continuous circuit.

In various embodiments, the circuit can further comprise the electrically conductive yarn knit in a vertical, horizontal, or angled direction in the fabric. In one embodiment, the electrically conductive yarn comprises a knit rib pattern to provide a vertical circuit direction in the fabric. In another embodiment, the electrically conductive yarn is knit along a course to provide a horizontal circuit direction in the fabric. To provide an angled circuit direction in the fabric, the electrically conductive yarn can be knit in a wale offset from a previous wale in successive courses. In yet other embodiments, the electrically conductive yarn can be laid in: a single course to provide a horizontal circuit direction; in a plurality of courses to provide an angled circuit direction; or in changing directions between courses to provide a multi-directional circuit direction.

In some embodiments, the wearable device can comprise an elastic fabric having an unstretched dimension in a direction of the circuit. In such an embodiment, stretch beyond the unstretched dimension in the circuit direction can be limited to provide sufficient circuit continuity for reliable conduction of the electrical signals. For example, when the circuit comprises a cut yarn, stretch is limited to about 5-10% beyond the unstretched dimension in the circuit direction. When the circuit comprises a continuously knit stretch nylon yarn, stretch is limited to about 10-20% beyond the unstretched dimension in the circuit direction. When the circuit comprises a continuously knit 70 denier spandex yarn, single or double covered with a conductive nylon yarn, stretch is limited to about 50-100% beyond the unstretched dimension in the circuit direction.

In certain embodiments, the location to which the electrical signal is transmitted comprises an external device separate from the wearable device. For example, the external device can comprise an electronic display unit configured to display the transmitted value of a variable.

In certain embodiments, the circuit can be configured to conduct electrical signals in both directions along the circuit. In particular embodiments, the circuit can be configured to transmit power from a power source to a location on the wearable device.

In some embodiments, the wearable device comprises a compressive pressure device, and the variable comprises compressive pressure applied by the device. In some embodiments, the wearable device comprises a compressive pressure device, a sensor is configured to sense compressive pressure in an area of a body to which the device is applied, and the transmission circuit is configured to transmit an electrical signal representing an amount of compressive pressure sensed in the area of a body to an external electronic display unit. In particular embodiments, the compressive pressure device comprises an inner compressive pressure sleeve and an overlying outer compressive pressure wrap. In such an embodiment, the sensor can be located either (a) between the body and the sleeve, (b) within the sleeve (c) between the sleeve and the wrap, or (d) within the wrap. In either of these locations, the sensor is configured to sense an actual cumulative amount of compressive pressure applied by the sleeve and the wrap.

Some embodiments of a compression and sensing system and method can include a wearable device comprising an elastic fabric; and a circuit for conducting electrical signals comprising an electrically conductive silver yarn or a yarn coated with silver knitted into the device fabric in a vertical, horizontal, or angled direction. In such embodiments, the circuit can further comprise (a) a sensor circuit configured to sense a variable in an area of a body to which the device is applied, and (b) a transmission circuit configured to transmit an electrical signal representing a value of the variable in the area of the body to an external electronic display unit configured to display the transmitted value of the variable. In some such embodiments, the device fabric has an unstretched dimension in a direction of the circuit, and stretch beyond the unstretched dimension in the circuit direction is limited to provide sufficient circuit continuity for reliable conduction of the electrical signals. In some such embodiments, the wearable device comprises a compressive pressure device, the variable comprises compressive pressure applied by the device, and the transmission circuit is configured to transmit an electrical signal representing an amount of compressive pressure sensed in the area of a body to an external electronic display unit.

Some embodiments of a compression and sensing system and method can include: a wearable device; a sensor configured to sense a variable in an area of a body to which the device is applied; and a transmission circuit comprising an electrically conductive yarn knitted into the device and configured to transmit an electrical signal representing a value of the variable in the area of a body to another location.

In some such embodiments, the sensor can further comprise a knitted cuff sensor. In one embodiment, the knitted cuff sensor comprises a three-layer capacitance type sensor comprising (a) an inner layer electrically conductive yarn, (b) a middle layer semi-conductive dielectric yarn, and (c) an outer layer electrically conductive yarn. In other such embodiments, the knitted cuff sensor comprises a two-layer capacitance type sensor comprising (a) an inner cuff layer and an outer cuff layer each comprising an electrically conductive yarn and (b) an electrically regulating dielectric material inserted between the inner and outer cuff layers. In yet other such embodiments, the knitted cuff sensor comprises a piezoelectric type sensor. In still other such embodiments, the knitted cuff sensor comprises a piezoresistive sensor comprising (a) an inner cuff layer and an outer cuff layer each comprising an electrically conductive silver yarn and (b) a piezoresistive semi-conductive polymer disposed between the inner and outer cuff layers.

In some embodiments, the compression and sensing system and method can further include a cuff integrally knit into the wearable device, in which the cuff is configured to house a sensor. The sensor can comprise an electro-mechanical sensor, a capacitance sensor, or a piezoelectric sensor. In some embodiments, the compression and sensing system and method can further include a pocket integrally knit into the wearable device, in which the pocket is configured to house the sensor. The sensor can comprise an electro-mechanical sensor, a capacitance sensor, or a piezoelectric sensor.

In some embodiments, the sensor can be securable to a hook-and-loop type fastener engagable with the wearable device. In such an embodiment, the sensor can comprise an electro-mechanical sensor, a capacitance sensor, or a piezoelectric sensor.

In some embodiments, the sensor can further comprise a sensor circuit printed onto a material comprising a hook-and-loop type fastener engagable with the wearable device. An electrically conductive yarn can be sewn through the material so that the yarn is conductively contactable between the printed sensor circuit and the transmission circuit in the wearable fabric.

In some embodiments, the wearable device comprises a compressive pressure device, the variable comprises compressive pressure, and the system can further comprise a pressurized cuff (a) having opposing ends releasably securable to each other, (b) adjustably positionable about the wearable device, and (c) having the sensor integrated into the cuff. When the pressurized cuff is adjusted about the wearable device to have the same initial compressive pressure as the wearable device, the sensor senses changes in actual applied pressure at an interface of the body area, the wearable device, and the pressurized cuff.

Some embodiments of the subject matter described herein include a compression and sensing system and method comprising a wearable compressive pressure device comprising an elastic fabric; an electrically conductive yarn knitted into the device and comprising a transmission circuit configured to transmit an electrical signal representing a compressive pressure value in an area of a body to a connection point on the transmission circuit; a sensor connectable to the transmission circuit and configured to sense compressive pressure in the area of a body to which the device is applied; and a data processor/display unit connectable to the transmission circuit and configured to display the transmitted compressive pressure value. The compressive pressure device can further comprise an inner compressive pressure sleeve having the transmission circuit knitted therein, and an outer compressive pressure wrap. The sensor can be further configured to sense compressive pressure applied by the inner sleeve and a cumulative compressive pressure applied by the inner sleeve and the outer wrap.

In some embodiments, the conductive yarn can further comprise a 70 denier conductive yarn having 24-68 filaments and a resistance between about 2-20 ohms per 10 cm along the transmission circuit. In some preferred embodiments, the conductive yarn is cut and laid in along the length of the compressive pressure sleeve. In some embodiments, the connection point on the transmission circuit is wider than the remainder of the transmission circuit so as to provide a more secure connection for the data processor/display unit.

In some embodiments, the sensor can further comprise a capacitive-type pressure sensor. In some embodiments, the sensor can further comprise a plurality of spaced apart projections extending sufficiently outward from the surface of the sensor to engage a patient's leg when attached to the inner compressive pressure sleeve, thereby evenly distributing force applied by the outer compressive pressure wrap onto the sensor. In some embodiments, the sensor can further comprise (1) two electrical connections extending in opposite directions from the sensor, each electrical connection configured to connect to a separate conductive yarn in the transmission circuit, and (2) an adhesive backing for adhering the sensor onto an outer surface of the compressive pressure device.

In some embodiments, the inner sleeve can further comprise a reciprocated heel pouch and an open toe, each adapted to guide placement of the inner sleeve and to maintain the inner sleeve in a therapeutic position on the body. As a result, wrinkling or bunching of the inner sleeve can be reduced so that the inner sleeve compacts evenly onto the body under compressive pressure exerted by the outer wrap.

Some embodiments of a compression and sensing method of the subject matter described herein include providing an inner compressive pressure sleeve having an electrically conductive yarn knitted therein to form a transmission circuit; applying the inner compressive pressure sleeve to a person's lower leg so that the transmission circuit is aligned along the sides of the lower leg; attaching a compressive pressure sensor to the conductive yarns in the transmission circuit at the smallest ankle circumference; connecting a data processor/display unit to connections points on the transmission circuit; reading on the data processor/display unit a first measurement of interface compressive pressure provided by the inner compressive pressure sleeve; beginning to wrap an outer compressive pressure wrap over the inner compressive pressure sleeve; when applying compression at the ankle, reading on the data processor/display unit a second measurement of the cumulative interface compressive pressure provided by the inner sleeve and the outer wrap; and adjusting the tightness of the outer wrap about the inner sleeve to adjust the cumulative interface compressive pressure.

In other embodiments of such a method, the sensor comprises an adhesive backing and two electrical connections extending in opposite directions from the sensor. The step of attaching a compressive pressure sensor to the conductive yarns in the transmission circuit can further comprise removing the adhesive backing from the sensor and adhering the sensor onto an outer surface of the inner sleeve; and connecting each electrical connection to a separate conductive yarn in the transmission circuit. In some embodiments, the outer compressive pressure wrap comprises a first and a second outer compressive pressure wrap. The method can thus further comprise beginning to wrap the second outer compressive pressure wrap over the first outer compressive pressure wrap. When applying compression at the ankle, a third measurement can be read on the data processor/display unit of the cumulative interface compressive pressure provided by the inner sleeve and the first and second outer wraps. Accordingly, the tightness of the second outer wrap can be adjusted about the first outer wrap to adjust the cumulative interface compressive pressure.

Features of a compression tension adjusting, stabilization, and sensing system and method of the subject matter described herein may be accomplished singularly, or in combination, in one or more of the embodiments of the subject matter described herein. As will be realized by those of skill in the art, many different embodiments of a compression tension adjusting, stabilization, and sensing system and/or method according to the subject matter described herein are possible. Additional uses, advantages, and features of the subject matter described herein are set forth in the illustrative embodiments discussed in the description herein and will become more apparent to those skilled in the art upon examination of the following.

### BRIEF DESCRIPTION OF THE DRAWINGS

FIG. 1 is a plan view of the sleeve of the sleeve-wrap compression system in position on a patient's lower leg in an embodiment of the compression and sensing system and method of the present invention.

FIG. 2 is a perspective view of the wrap of the sleeve-wrap compression system in rolled form ready to be applied to a patient's limb in an embodiment of the compression and sensing system and method of the present invention.

FIG. 3 is a plan view of the wrap in FIG. 2 overlapped onto itself after being applied over the sleeve (not shown) on a patient's lower leg in an embodiment of the compression and sensing system and method of the present invention.

FIG. 4 is a graphic view of the first shade of brown representing the first length (or light) stretch, the second shade of brown representing the second length (or medium) stretch, and the third shade of brown representing the third length (or firm) stretch in the color/compression change indication system in an embodiment of the compression and sensing system and method of the present invention.

FIG. 5 is a plan view of the wrap in FIG. 2 positioned on a foot and lower leg, with sufficient tension so that the wrap consistently exhibits the third shade of brown in an embodiment of the compression and sensing system and method of the present invention.

FIG. 6 is a graphic view of a high slope value, or steep stress/strain curve, of a stiff compression garment.

FIG. 7 is a graphic view of a stress/strain curve of a moderately stiff compression device.

FIG. 8 is a graphic view of the more gradual slope value, or stress/strain curve, of the sleeve-wrap compression system in an embodiment of the compression and sensing system and method of the present invention.

FIG. 9 is a graphic view of data points showing that the sleeve-wrap system maintains working compression within a desired range for seven days while the system is being worn.

FIG. 10 is a graphic view illustrating anti-microbial action by copper in the wrap and by silver in the sleeve, the presence of hydrophilic wicking fibers in the sleeve and in the wrap, and vertical wicking of moisture/exudate through the sleeve layer and through the wrap layer to the surface of the wrap layer in embodiments of the compression and sensing system and method of the present invention.

FIG. 11 is a view of a body monitoring system on a lower limb of a wearer in an embodiment of the compression and sensing system and method of the present invention.

FIG. 12 is a view of a body monitoring system having knitted-in sensing and transmission circuits in an embodiment of the compression and sensing system and method of the present invention.

FIG. 13 is a view of a body monitoring system having a knitted-in cuff and transmission circuit in an embodiment of the compression and sensing system and method of the present invention.

FIG. 14 is a diagrammatic view of an electrically conductive yarn knitted as an angled transmission circuit in an embodiment of the compression and sensing system and method of the present invention.

FIG. 15 is a diagrammatic view of an electrically conductive yarn laid in a knitted fabric structure as a transmission circuit in an embodiment of the compression and sensing system and method of the present invention.

FIG. 16 is a view of a body monitoring system having a knitted-in pocket in an embodiment of the compression and sensing system and method of the present invention.

FIG. 17 is a view of a compressive pressure device having a knitted-in cuff and transmission circuit in an embodiment of the compression and sensing system and method of the present invention.

FIG. 18 is a view of a piece of material having a printed sensor circuit, engaged with a wearable fabric with a hook-and-loop type fastener, and conductively connected to a transmission circuit in the fabric in an embodiment of the compression and sensing system and method of the present invention.

FIG. 19 is a view of an adjustable pressurized sensor cuff and a transmission circuit connecting the sensor cuff to a display unit in an embodiment of the compression and sensing system and method of the present invention.

FIG. 20 is a view of an inner compression sleeve having integrally knit sensing and transmission circuits and an overlying compression wrap in an embodiment of the compression and sensing system and method of the present invention.

FIG. 21 is a front view of a compression device sleeve having a transmission circuit in an embodiment of the compression and sensing system and method of the present invention.

FIG. 22 is a photographic perspective view of a compression device sleeve having a transmission circuit in an embodiment of the compression and sensing system and method of the present invention.

FIG. 23 is a diagrammatic top view of a pressure sensitive sensor in an embodiment of the compression and sensing system and method of the present invention.

FIG. 24 is a photographic front perspective view showing application of a pressure sensitive sensor to a transmission circuit in an embodiment of the compression and sensing system and method of the present invention.

FIG. 25 is a photographic front perspective view showing connection of leads from a pressure reader and display device to a transmission circuit in an embodiment of the compression and sensing system and method of the present invention.

FIG. 26 is a photographic side view showing application of the wrap of the sleeve-wrap compression system over the sleeve and attached pressure sensor in an embodiment of the compression and sensing system and method of the present invention.

FIG. 27 is a photographic top view showing application of the wrap of the sleeve-wrap compression system over the top portion of the sleeve in an embodiment of the compression and sensing system and method of the present invention.

FIG. 28 is a diagrammatic view of an electrically conductive yarn laid in a jersey knit fabric structure as part of a transmission circuit in an embodiment of the compression and sensing system and method of the present invention.

FIG. 29 is a table showing results comparing measurements of compressive pressure applied by the outer compressive pressure wrap as shown in FIGS. 2 and 3, the measurements taken by (1) a data processor in an embodiment of a compression and sensing system of the present invention, and (2) a PICOPRESS® compression measurement device.

FIG. 30 is a graph showing the results of the comparative measurements of compressive pressure shown in FIG. 29.

FIG. 31 is a graph depicting the approximate linear relationship between stretch (stress) on the x-axis and pressure (strain) on the y-axis in a stress-strain curve for embodiments of the tension adjusting and stabilization system and method of the present invention in which the elastic compression bandage has a 1:1 stretch characteristic.

FIG. 32 is a diagrammatic view of a repeating series of lines across at least a part of the width of the elastic compression bandage at predetermined spaced-apart intervals along the length of the bandage and labeled with matching numbers in an embodiment of the tension adjusting and stabilization system and method of the present invention.

FIG. 33 is a diagrammatic view of the elastic compression bandage having a tension pressure color indicator along the length of the bandage in an embodiment of the tension adjusting and stabilization system and method of the present invention.

FIG. 34 is a diagrammatic view of one side of the elastic bandage comprising one tension pressure indicator color in an embodiment of the tension adjusting and stabilization system and method of the present invention.

FIG. 35 is a diagrammatic view of the elastic bandage comprising a tension pressure color indicator and including a spaced-apart, repeating series of oval shapes along its length in an embodiment of the tension adjusting and stabilization system and method of the present invention.

FIG. 36 is a diagrammatic view of the elastic bandage having a tension pressure color indicator comprising a spaced-apart, repeating series of colored square shapes along its length in an embodiment of the tension adjusting and stabilization system and method of the present invention.

FIG. 37 is a diagrammatic view of the elastic compression bandage comprising a set of increasingly larger, dark-colored rectangular boxes having number representing an increasingly larger amount of compressive pressure obscured within each that becomes visible when the rectangular boxes are stretched to become squares in an embodiment of the tension adjusting and stabilization system and method of the present invention.

FIG. 38 is a diagrammatic view of the elastic compression bandage having a tension pressure indicator comprising a rectangle shape with an oval inside in an unstretched position that changes to a square shape having a circle inside when a particular compressive pressure is achieved in an embodiment of the tension adjusting and stabilization system and method of the present invention.

### DESCRIPTION

This application claims benefit of U.S. Provisional Patent Application No. 62/631,675, filed Feb. 17, 2018, and is a Continuation-in-Part application of U.S. patent application Ser. No. 15/371,627, filed Dec. 7, 2016, which: (1) claims benefit of U.S. Provisional Patent Application No. 62/264,244, filed Dec. 7, 2015; (2) is a Continuation-in-Part application of U.S. patent application Ser. No. 14/225,952, filed Mar. 26, 2014, which claims benefit of U.S. Provisional Patent Application No. 61/805,175, filed Mar. 26, 2013; and (3) is a Continuation-in-Part application of U.S. patent application Ser. No. 14/098,730, filed Dec. 6, 2013. Each of these applications is incorporated by reference herein in its entirely.

For the purposes of this description, unless otherwise indicated, all numbers expressing quantities, conditions, and so forth used in the description are to be understood as being modified in all instances by the term “about.” Accordingly, unless indicated to the contrary, the numerical parameters set forth in the following description are approximations that can vary depending upon the desired properties sought to be obtained by the embodiments described herein. At the very least, and not as an attempt to limit the application of the doctrine of equivalents to the scope of the invention, each numerical parameter should at least be construed in light of the number of reported significant digits and by applying ordinary rounding techniques.

Notwithstanding that the numerical ranges and parameters setting forth the broad scope of the described embodiments are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10.

As used in this description, the singular forms “a,” “an,” and “the” include plural referents unless the context clearly dictates otherwise. Thus, for example, the term “a yarn” is intended to mean a single yarn or more than one yarn. For the purposes of this specification, terms such as “forward,” “rearward,” “front,” “back,” “right,” “left,” “upwardly,” “downwardly,” and the like are words of convenience and are not to be construed as limiting terms. Additionally, any reference referred to as being “incorporated herein” is to be understood as being incorporated in its entirety.

Embodiments of the subject matter described herein include a compression tension adjusting, stabilization, and sensing system and method. Embodiments of the compression tension adjusting, stabilization, and sensing system and method can comprise one or more of the following components: a compression and sensing system and method; a sleeve-wrap compression system and method; a body monitoring system and method; and a tension adjusting and stabilization system and method.

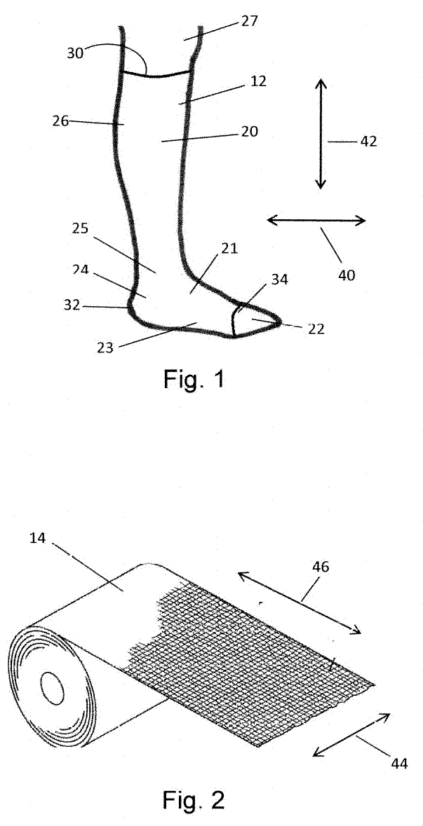

Some embodiments of the subject matter described herein include a compression and sensing system and method comprising a sleeve-wrap compression system and method. FIGS. 1-10 illustrate such embodiments. Embodiments of the sleeve-wrap compression system 10 and/or method can comprise multiple compressive pressure layers. The first layer for applying adjacent a patient's skin is a compressive pressure sleeve 12. The second layer that is applied to the top of the first, sleeve layer comprises a compressive pressure wrap 14. Embodiments of the sleeve-wrap compression system 10 can include one or more compressive pressure wrap layers 14 on top of the sleeve 12. Preferred embodiments of the multi-layer sleeve-wrap compression system 10 comprise a two-layer system comprising a sleeve layer 12 and a single wrap layer 14. The system 10 is useful for the treatment and management of venous leg ulcers and/or other complications of venous incompetency. Certain embodiments of the sleeve-wrap compression system 10 can be utilized for treatment of lymphedema and/or other edematous conditions of body extremities.

It has been found that the combination of the inner sleeve layer 12 and the outer wrap layer 14 according to embodiments of the sleeve-wrap compression system 10 provide particular desirable and advantageous features for effective wound treatment. For example, the sleeve and wrap layers 12, 14, respectively, are easy to properly apply by professional and lay caregivers. Once applied, the sleeve-wrap compression system 10 can be reliably maintained in a desired position on a patient's limb 20 with minimal slippage. It was found that the sleeve-wrap compression system 10 provides consistent compressive pressure during an extended wear period, for example, over 5-7 days. In contrast to stiffer compression systems, the sleeve-wrap system 10 provides a controlled, gradual change in applied compressive pressure in response to a change in limb volume. Quite advantageously for effective wound treatment, the sleep-wrap compression system 10 provides consistent compressive pressure during varying degrees of patient activity and rest. Thus, the sleeve-wrap compression system 10 is able to control applied compressive pressure so as to maintain a consistent working compression profile. As a result, the sleeve-wrap compression system 10 can maintain an optimal, therapeutic level of compressive pressure for the treatment of leg ulcers, for example, over time.

The sleeve layer 12 of the sleeve-wrap compression system 10 comprises a tubular sleeve similar to a compression stocking or hosiery. FIG. 1 shows the sleeve 12 in place on a patient's lower leg 20. The sleeve 12 is designed to be slid over a patient's limb 20, such as over a foot 21, toe 22, instep 23, heel 24, ankle 25, and calf 26, or over a hand and arm. Various embodiments of the sleeve 12 can be configured to cover different lengths of a limb 20. Typically, a lower limb sleeve 12 can extend from the foot 21 to just below the wearer's knee 27. The sleeve layer 12 can be fabricated with a variety of materials suitable for application on the skin and for providing compressive pressure. In preferred embodiments, the sleeve layer 12 comprises spandex in combination with nylon, acrylic, polyester, and/or cotton.

One aspect of the present invention is that the sleeve layer 12 provides a smooth dispersal of compression over the limb 20 to which it is applied. The sleeve layer 12 of the sleeve-wrap compression system 10 can be made in a seamless manner. In some preferred embodiments, the sleeve 12 is constructed to include a knitted terry lining 30 on the entire inner, skin facing surface of the sleeve 12. A knitted terry fabric 30 is a plated fabric knitted with two different yarns. A ground yarn appears on one side of the fabric, and a looped, or effect, yarn is pulled out the other, technical side of the fabric to make a looped or pile texture. The seamless, terry lined sleeve 12 provides a smooth surface next to the skin in which there is no overlapping of fabric. The beneficial effect is that there are no hard compression lines or creases on the skin due to fabric overlapping or from edges of the sleeve 12 on the limb 20. As a result, the sleeve 12 provides a smooth dispersal of compression over the limb 20 from the overlying wrap 14. This smooth application of compression helps protect already compromised skin, prevent further skin breakdown in areas adjacent the wound, and enhance compressive pressure therapy. Thus, the sleeve layer 12 of the sleeve-wrap compression system 10 provides an advantage over conventional multi-layer compression systems in which overlapping of fabric in the wrap adjacent a patient's skin causes creases in the skin, thereby promoting skin breakdown. In addition, the interior terry lining 30 in the sleeve layer 12 provides a soft cushioning that enhances comfort of the wearer.

The yarn comprising the terry lining 30 can comprise a thermally adaptive yarn, such as a yarn incorporating Outlast® technology, that goes through phase changes to control temperature (available commercially from Outlast Technologies LLC, 831 Pine Ridge Road, Golden Colo. 80403). Such technology utilizes phase change materials that absorb, store, and release heat for enhanced thermal comfort. As the skin gets hot, the heat is absorbed by microencapsulated phase change materials, and as it cools, that heat is released. In this way, heat is managed proactively and the production of moisture is controlled before it begins. Accordingly, fabric that incorporates this type of yarn results in decreased sweating by the wearer. Thus, such temperature responsive fabric in the sleeve 12 provides the advantages of enhancing patient comfort and, by helping to keep the wound dry in warm conditions and decreasing vasoconstriction in cooler conditions, enhances wound healing as well.

Another aspect of the present invention is that the sleeve layer 12 stays in place on a patient's limb 20. Maintaining a compression system in proper position on a patient's limb is critical to provide accurate compressive pressures to the limb/wound. One of the biggest disadvantages of compression systems that utilize only wraps is that the wraps do not reliably stay in place.

In embodiments of the sleeve 12 having an interior terry lining 30, the soft, textured quality of the terry lining 30 provides a particularly desirable and effective gripping onto the skin of a patient, which minimizes any tendency of the sleeve towards slippage after application. That is, the terry lining 30 of the sleeve 12 helps keep the sleeve 12 in a desired position about a patient's limb 20.

In some embodiments, the sleeve 12 can be constructed to have the anatomical contour of a limb 20. Such shaped construction of the sleeve 12 can be accomplished by manipulating the knitting program so as to control tension of the spandex and elongation of the stitch to produce a contoured shape. An anatomically contoured sleeve 12 provides a more snug fit onto the limb 20. As a result, wrinkling and/or bunching of sleeve fabric, for example, on the top of the foot 21, is significantly reduced or eliminated. This decreases the risk that skin irritation or adverse pinpoint pressure on the wound would occur as a result of fabric creases in the sleeve 12. In this way, the sleeve 12 protects bony prominences. In addition, without extra fabric in the folds and crevices of a limb, an anatomically contoured sleeve allows the sleeve 12 to compact evenly onto the limb 20 under pressure from the overlying wrap 14. Thus, under compressive pressure from the wrap 14, the sleeve 12 remains both smooth and in place.

In particular, in some embodiments, the sleeve 12 can include a reciprocated heel pocket or pouch 32. A reciprocated heel 32 is formed by the three-dimensional shaping of a pouch, achieved on a small-diameter hosiery knitting machine by using held loop shaping so that the number of courses knitted by adjacent needles is varied in order to knit a pouch for the heel. During pouch knitting, the rotating movement of the cylinder changes to a semi-circular or oscillatory (reciprocated) movement using selected needles to produce the heel pouch 32. The reciprocated heel pouch 32 allows the sleeve to have a more formed fit about the heel 24 of a wearer. As a result, the reciprocated heel 32 in the sleeve 12 helps ensure proper positioning of the sleeve 12 about the limb 20, thereby helping to reduce wrinkling and/or bunching of fabric on the top of the foot 21 and elsewhere.

In some embodiments, the sleeve 12 can be constructed to include an open toe 34. The open toe 34 provides further ability to apply the sleeve 12 in a desired position about the foot 21 and lower limb 20. Moreover, the open toe 34 ensures that the patient's toes 22 are not being compressed, and allows easy access to assess vascular supply and condition of the forefoot.

In some embodiments, the sleeve 12 and the wrap 14 can each be constructed so that the interior of the wrap 14 and the exterior of the sleeve 12 exert a desirably enhanced amount of friction between them when the wrap 14 is applied to the sleeve 12. An enhanced friction co-efficient between the sleeve 12 and the wrap 14 helps to maintain the wrap 14 in position on the sleeve 12, thereby decreasing the potential for downward slippage and helping to maintain the entire sleeve-wrap system 10 in proper position on the limb 20. As a result, the risk of skin irritation from displaced compression layers is reduced and the delivery of consistent compressive pressure for optimal wound healing is enhanced.

In some embodiments, the sleeve 12 comprises a construction that permits horizontal stretch 40 with minimal vertical, or longitudinal, stretch 42. Horizontal stretch 40 creates tension around the circumference of the limb 20, which helps keep the sleeve 12 up on the limb 20 and thus avoid undesirable slippage. An ability to stretch to a large degree vertically 42 (along the longitudinal axis of a limb) creates the potential for a garment to slip downward. To provide minimal vertical stretch, the sleeve 12 can be constructed so as to pack stitches in the vertical direction, which causes the knitted fabric to resist stretching in the vertical direction 42. In some embodiments, such a construction comprises spandex yarns “laid in” horizontally into the knit structure without formation of stitches or loops to hold the spandex. In a “laid in” fabric, a base structure of knitted or overlapped threads hold in position other non-knitted threads which are incorporated, or “laid in,” into the structure during the same knitting cycle. Although an inlaid yarn is not formed into a knitted loop, the base fabric structure can utilize various knitting stitches to hold the inlaid yarn in place. Laying in spandex yarns horizontally in the sleeve allows horizontal stretch 40, while avoiding an additional course of interlocking loops that permit stretch in the vertical direction 42. Thus, as compared to an approximately 100% vertical stretch 42 resulting from knitted spandex yarns, horizontally laid-in spandex yarns can reduce vertical stretch 42 in the sleeve 12 to about 30%.

The sleeve 12 of the sleeve-wrap compression system 10 can be constructed so that the horizontal stretch 40 provides a small, uniform amount of compressive pressure throughout the length of the sleeve 12. For example, in preferred embodiments, the sleeve 12 can provide about 5-10 mm Hg of compressive pressure along the length of the sleeve 12. A small amount of compressive pressure allows the sleeve 12 to be sufficiently elastic so as to grip the contours of the limb 20 to which it is applied and help maintain the sleeve 12 in its original position over time. In contrast, each of the layers in conventional multi-layer compression systems comprises a wrap. Over time, the multiple wraps tend to move up and/or down along a patient's limb and thus become more loosely (or more tightly) wrapped about the limb. As a result, such conventional multi-layer wrap systems can lead to undesirably varying amounts of compressive pressure on the limb. However, a small, uniform amount of compressive pressure in embodiments of the sleeve 12 of the sleeve-wrap compression system 10 helps keep the sleeve 12 in a desired position.

Similarly, a small amount of compressive pressure allows the sleeve 12 to be sufficiently elastic with respect to changes in limb circumference due to edema that the sleeve 12 can provide a consistent, uniform compressive pressure in response to such change. That is, with reference to the description related to FIGS. 6-8, the sleeve 12 can be constructed so that its elasticity exhibits a relatively flat stress/strain curve. As the sleeve is stretched/stressed, even to a large degree, by increasing limb circumference, the amount of compressive pressure (strain) applied to a patient's limb 20 remains within a controlled, narrow range. In this way, the sleeve 12 overcomes the problem of varying pressures in conventional multi-layer wrap systems by providing a consistent, uniform amount of compressive pressure along the length of the sleeve 12 over time.

In addition, the amount of compressive pressure provided by the underlying sleeve 12 serves to limit the amount of pressure that the overlying wrap 14 must provide to reach a particular cumulative pressure. Thus, a single wrap 14 can be constructed to exert a lesser amount of pressure, which makes the wrap 14 easier to apply.

Each of these aspects of the sleeve-wrap compression system 10 individually, and particularly in combination, helps keep the sleeve 12 in a desired position on a limb 20 so that a stable compressive pressure can be maintained by the sleeve 12 and the overlying wrap 14. In addition, such features in the sleeve 12 provide a smooth dispersal of compression from the overlying wrap 14, thereby further enhancing control of compressive pressure onto the limb 20 so as to optimize treatment of venous ulcers.

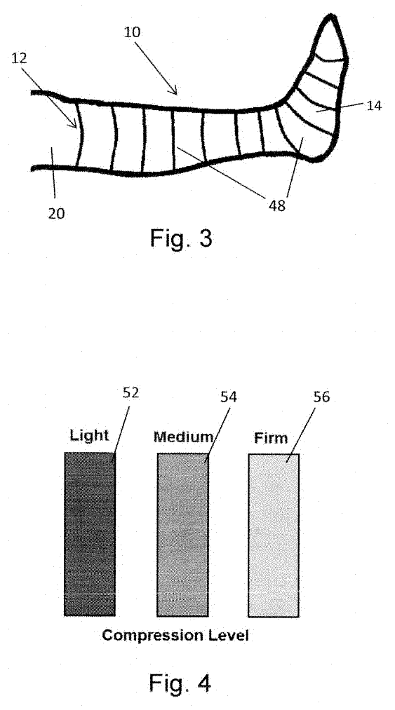

Embodiments of the wrap layer 14 of the sleeve-wrap compression system 10 can comprise an elongated elastic wrap, or bandage. FIG. 2 shows the wrap 14 in rolled form ready to be applied to a patient's limb 20. The wrap 14 preferably includes spandex in combination with nylon and/or cotton. In some preferred embodiments, the wrap 14 comprises a width 44 of about four inches. It was discovered that the wrap 14 that is four inches wide stays in place on the underlying sleeve 12 without slippage better than a three-inch wrap, particularly in the heel region 24. Preferably, the wrap 14 comprises a sufficient length so that when the wrap is applied with a 50% overlap 48 onto itself the wrap 14 covers the length of the underlying sleeve 12 on a patient's limb 20. FIG. 3 shows the wrap 14 overlapped 48 onto itself after being applied over the sleeve 12 (not shown) on a patient's lower leg 20.

In some embodiments, the wrap 14 can comprise a material in which at least the exterior surface has one portion of a hook-and-loop type fastener that is engagable with a mating portion of such a fastener. In this way, after the wrap 14 is applied, it can be secured to itself with one or more strips of a mating portion of the hook-and-loop type fastener. The hoop-and-loop fastening capability is advantageous for securing the wrap 14, as opposed to metal clips that can be uncomfortably bulky or tape that is susceptible to slippage from moisture. When the hook-and-loop fastening enabled wrap 14 is being applied onto a patient's limb, one overlapping portion of the wrap 14 is adhered to another underlapping portion 14. In this way, the wrap 14 can be secured onto itself about the anatomical contours of the limb 20, such as about a patient's heel 24. Such contoured securing of the wrap 14 helps maintain the wrap 14, and the sleeve-wrap compression entity 10, in a desired therapeutic position on the limb 20. In certain embodiments, pieces of a mating portion of a hook-and-loop type fastener can be adhered to one or more areas on the hook-and-loop fastening enabled wrap 14 to create a smooth surface on the wrap 14. For example, pieces of a mating portion of a hook-and-loop type fastener can be adhered to the wrap at the back of the heel 24 and/or on top of the foot 21 to create smooth, anti-friction areas.

Various wraps 14 can be constructed to provide different amounts of compressive pressure. The amount of compressive pressure a particular wrap 14 will provide depends on stretch characteristics selected during construction of the wrap 14 and the amount of stretch applied to the wrap 14 while it is being overlaid onto the sleeve 12. The amount of compressive pressure therapy desired depends on the clinical use of the wrap 14 and the individual patient. For example, the wrap layer 14 of the sleeve-wrap compression system 10 designed for treatment of a leg ulcer may provide compressive pressure at the instep/ankle area 23/25 in the range of about 10-60 mm Hg, preferably in the range of about 20-45 mm Hg, and may provide compressive pressure at the calf area 26 in the range of about 10-60 mm Hg, preferably in the range of about 15-40 mm Hg. One embodiment of the outer wrap layer 14 that is particularly useful in the treatment of venous leg ulcers is configured to provide between about 5-10 mm Hg compressive pressure and about 30-35 mm Hg compressive pressure depending on the amount of longitudinal stretch 46 applied to the wrap 14.

The sleeve layer 12 can provide a uniform, low level compression, for example, about 5 mm Hg of compressive pressure. Therefore, such preferred embodiments of the sleeve-wrap compression system 10 can provide cumulative compressive pressures at the instep/ankle area 23/25 in the range of about 25-50 mm Hg, and at the calf area 26 in the range of about 20-45 mm Hg. The cumulative applied compressive pressure in the sleeve-wrap compression system 10 may be a uniform amount throughout the length of the system 10, or may be graduated from a larger pressure at the instep/ankle area 23/25 to a smaller pressure at the calf area 26. In an embodiment of the sleeve-wrap compression system 10 intended for use with lymphedema, the cumulative applied compressive pressure can be as high as 100 mm Hg at the ankle 25.

One of the benefits of utilizing the sleeve-wrap compression system 10 in wound care is that the compressive pressure helps decrease edema. In some embodiments of the sleeve-wrap compression system 10, the wrap portion 14 comprises stretch characteristics that help control changes in applied compressive pressure as edema is reduced and the volume of a limb 20 changes. The stretch characteristics in such a wrap 14 having defined stretch-compressive pressure relationships are provided by a balance of several factors, including (1) size or denier of spandex; (2) stretch characteristics of spandex; and (3) the number of ends per unit of measure, or density, of spandex in the wrap fabric. For example, in some embodiments, the denier of spandex can vary from about 20 denier to about 600 denier, preferably from about 350 denier to about 500 denier. In some embodiments, the spandex-comprising wrap 14 can stretch 46 to about 400% greater than its relaxed length, preferably to about 200% greater than its relaxed length. In some embodiments, the wrap 14 can comprise from about 5 ends to about 50 ends of spandex per inch, preferably from about 5 ends to about 20 ends per inch.

In some preferred embodiments, the wrap 14 has a maximum stretch 46 of about 165% greater than its relaxed length and a clinically usable stretch 46 of about 30% to about 100% greater than its relaxed length. In particularly preferred embodiments, when the wrap 14 is stretched to a first, about 30% greater length, the compressive pressure applied to an exemplary nine-inch circumference is about 5-10 mm Hg. When the wrap 14 is stretched to a second, about 75% greater length, the compressive pressure applied to an exemplary nine-inch circumference is about 20 mm Hg. And when the wrap 14 is stretched to a third, about 100% greater length, the compressive pressure applied to an exemplary nine-inch circumference is about 30-35 mm Hg. That is, the compressive pressure applied by the wrap 14 in such preferred embodiments can range about 20-30 mm Hg pressure from a light stretch (the first, about 30% stretch) of the wrap to a firm stretch (the third, about 100% stretch) of the wrap 14.

These references to stretch of the wrap 14 refer to lengthwise extension of the wrap 14, or “vertical” (longitudinal) stretch 46. In some embodiments, the wrap 14 can be constructed to have vertical, or longitudinal, stretch 46 (that is, in the warp direction) with minimal horizontal stretch, or cross-stretch 44 across the width of the wrap 14 (that is, in the weft direction). Minimization of cross-stretch 44 in the wrap 14 helps conform the wrap 14 to the curvature of a patient's limb 20, thereby reducing the possibility of the wrap 14 producing any fabric folds around anatomical structures of the limb 20.

Such predetermined stretch characteristics in embodiments of the sleeve-wrap compression system 10 allow the wrap 14 to be stretched a particular amount to provide compressive pressure levels within a prescribed range. Applying and maintaining accurate compressive pressure helps ensure that a desired level of therapy for a wound is achieved.

Embodiments of the compression system 10 of the present invention can further comprise a color/compression change indication system 50 in which a particular amount of stretch of the wrap 14 creates a unique color, or shade of color, representative of a particular amount of compressive pressure. To accomplish a change in color, or shade, the wrap is fabricated with an intended “grin,” or “grin-through,” capability. Grin/grin-through is defined as the appearance of an interior layer of material when a fabric is stretched. For example, a core yarn having one color can be covered with a covering yarn having a different color. When a fabric comprising the differently colored core and cover yarns is stretched, the turns of the cover yarn can separate so that the core yarn is exposed through the cover yarn. The amount of separation of the cover yarn is directly related to the degree to which the fabric/yarn is stretched. Thus, the more a fabric is stretched, the more the turns of the cover yarn separate, resulting in a greater amount of grin-through of the core yarn color. Likewise, the more a fabric is stretched, the greater the change in color or shade of the fabric.

As applied to the sleeve-wrap compression system 10, some embodiments of the wrap 14 can comprise an elastic material having one color, or shade, in an unstretched condition that changes to a different color, or shade, in a stretched condition. The different, stretched color corresponds to a predetermined amount of stretch applied to the material, which in turn corresponds to a predetermined amount of compressive pressure. The stretched color can comprise a first stretched color corresponding to a first predetermined amount of stretch and a second stretched color corresponding to a second predetermined amount of stretch. The first amount of stretch and the second amount of stretch can each correspond to a different predetermined amount of compressive pressure.

For example, the wrap 14 can comprise a covering yarn comprising a covering yarn color and wrapped a number of turns about an elastic yarn comprising an elastic yarn color different than the covering yarn color. When the wrap 14 is stretched a first amount, the turns of the covering yarn move apart from each other to expose a first amount of the elastic yarn color corresponding to a first predetermined amount of compressive pressure. Likewise, when the wrap 14 is stretched a second amount, the turns of the covering yarn move apart from each other to expose a second amount of the elastic yarn color corresponding to a second predetermined amount of compressive pressure. That is, each of different amounts of wrap stretch can provide a unique color profile of a different combination of the covering yarn color and the elastic yarn color. Each unique color profile can correspond to a different amount of compressive pressure.

In one embodiment of the sleeve-wrap compression system 10, the wrap 14 comprises yarns have a core yarn that is white and a covering yarn that is brown. In a relaxed, unstretched state, the wrap 14 exhibits the brown color of the cover yarn. When the wrap 14 is stretched to a first length that is about 30% greater than its relaxed length, a first amount of the white color of the core yarn grins through the cover yarn to exhibit a first shade of brown 52 that is lighter than the “undiluted” brown of the cover yarn. When the wrap 14 is further stretched to a second length that is about 75% greater than its relaxed length, a second, greater amount of the white color of the core yarn grins through the cover yarn to exhibit a second shade of brown 54 that is even lighter than the first shade of brown 52. When the wrap 14 is further stretched to a third length that is about 100% greater than its relaxed length, a third, still greater amount of the white color of the core yarn grins through the cover yarn to exhibit a third shade of brown 56 that is even lighter than the second shade of brown 54. FIG. 4 shows the first shade of brown 52 representing the first length (or light) stretch, the second shade of brown 54 representing the second length (or medium) stretch, and the third shade of brown 56 representing the third length (or firm) stretch.

The shade of color produced by a certain amount of fabric stretching in the wrap 14 is associated with a particular level of compressive pressure. For example, in some preferred embodiments, when the wrap 14 is stretched to the first, about 30% greater length, the compressive pressure applied to an exemplary nine-inch circumference is about 5-10 mm Hg. When the wrap 14 is stretched to the second, about 75% greater length, the compressive pressure applied to an exemplary nine-inch circumference is about 20 mm Hg. And when the wrap 14 is stretched to the third, about 100% greater length, the compressive pressure applied to an exemplary nine-inch circumference is about 30-35 mm Hg. Accordingly, when the wrap 14 is applied to an exemplary nine-inch circumference with the first, about 30% stretch, the first shade of brown 52 exhibited by the wrap 14 represents a compressive pressure of about 5-10 mm Hg. With the second, about 75% stretch, the second shade of brown 54 exhibited by the wrap represents a compressive pressure of about 20 mm Hg. And with the third, about 100% stretch, the third shade of brown 56 exhibited by the wrap 14 represents a compressive pressure of about 30-35 mm Hg. That is, the compressive pressure applied by the wrap 14 in such preferred embodiments can range about 20-30 mm Hg pressure from a light stretch (the first, about 30% stretch) of the wrap 14 to a firm stretch (the third, about 100% stretch) of the wrap 14.

In an alternative embodiment, the sleeve-wrap compression system 10 can include a color/compression change indication system 50 in which a particular amount of stretch of the wrap 14 reveals a unique indicator, such as a particular shape or design, representative of a particular amount of compressive pressure. The indicator can comprise one or more indicia knitted into, or printed onto, the wrap 14. For example, the wrap 14 can include a first indicium comprising a rectangle having a first length that represents a first amount of stretch and a corresponding first predetermined amount of compressive pressure. Stretching the wrap 14 a second, greater amount of stretch causes the appearance of a second indicium comprising a rectangle having a second length that is shorter than the first length. The second indicium uniquely represents the second amount of stretch and a corresponding second predetermined amount of compressive pressure that is greater than the first amount of compressive pressure. Stretching the wrap 14 a third, even greater amount of stretch causes the appearance of a third indicium comprising a rectangle having a third length that is shorter than the second length. The third indicium uniquely represents the third amount of stretch and a corresponding third predetermined amount of compressive pressure that is greater than the second amount of compressive pressure. In such an embodiment, each of different amounts of wrap stretch can provide a unique indicium that represents a different amount of stretch and a corresponding different amount of compressive pressure. In this way, a user of the sleeve-wrap compression system 10 can readily determine a proper amount of stretch in the wrap 14 for providing a desired amount of compressive pressure.

The amount of compressive pressure applied by a compression garment to a limb depends in part on the circumference, or radius, of the limb. It has been proposed that pressure provided by compression hosiery on a limb can be characterized by Laplace's Law for cylindrically-shaped objects, expressed as P=T/r, where P is the internal pressure of the limb, T represents the wall tension across a slice of a cylindrical portion of hosiery, and r is the radius of the limb (the limb is approximated as a cylinder). Laplace's Law implies that the pressure supplied by compression hosiery varies inversely with the radius of the limb. In other words, if tension is equal throughout the garment, less pressure will be provided at a larger radius portion of the limb, such as the calf, than at a smaller radius portion of the limb, such as the ankle.

With respect to this inverse relationship between compressive pressure and limb radius, embodiments of the sleeve-wrap compression system 10 can be applied so as to provide desirably graduated compressive pressure from a distal point to a proximal point up a limb 20. As described herein, the sleeve 12 can be fabricated to provide the same small amount of compressive pressure, for example, 5 mm Hg pressure, along its length. By applying the wrap 14 under the same tension, that is, with the same amount of stretch, over the entire length of the sleeve 12, more compressive pressure will be provided at the smaller distal portions of the limb 20 and less compressive pressure will be provided at the larger proximal portions of the limb 20. In this way, the compressive pressure along the limb 20 will be graduated as desired.

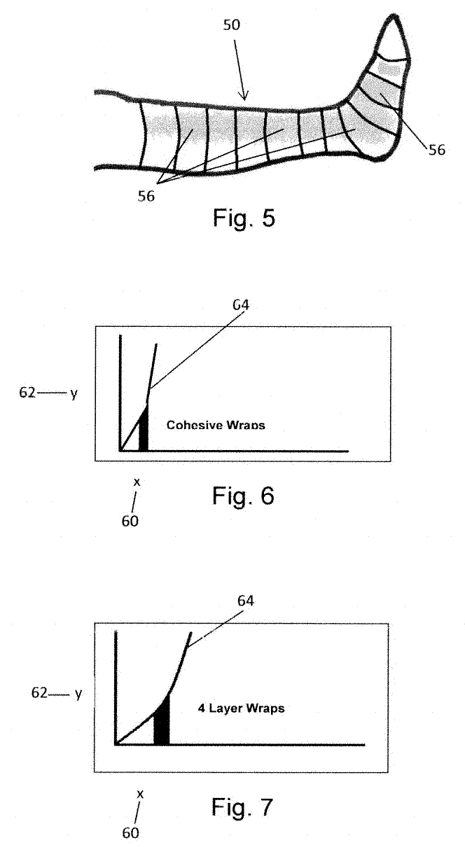

The relatively same tension, or amount of stretch, along the length 46 of the wrap 14 can be readily accomplished by applying the wrap 14 so that the same shade of color is exhibited throughout the wrap 14. As shown in the example in FIG. 5, in one embodiment, the sleeve 12 is positioned on a foot 21 and lower leg 20. Then a four-inch wide wrap 14 is applied over the sleeve 12 so that the wrap 14 has a 50% overlap onto itself, with sufficient tension so that the wrap 14 consistently exhibits the third shade of brown 56. As a result, the wrap 14 is stretched to the third, about 150% stretch that provides a uniform compressive pressure of about 30-35 mm Hg. The compressive pressure at the distal area of the foot 21 (from the sleeve 12 and wrap 14 together) is thus about 30-35 mm Hg. Since the leg 26 has a larger circumference than the foot 21 and increases in circumference from the ankle 25 to the knee 27, the compressive pressure graduates in a decreasing fashion proximally along the leg 20 such that the compressive pressure at the knee 27 is less than at any other area in the foot 21 or leg 20. Thus, maintaining the same color of the wrap 14 along the leg 20 allows the user to control the amount of compressive pressure being applied. Accordingly, the sleeve-wrap compression system and/or method 10 help ensure a proper desired graduated pressure along the limb 20.

In addition, maintaining the same color or shade of the wrap 14 along the limb 20 to provide a uniform amount of applied compressive pressure allows changes in compression levels along the limb 20 to be smooth even as a reduction in edema causes a decrease in limb girth. That is, maintaining the same applied compressive pressure along the limb 20 ensures that as edema and limb girth are reduced, the compressive pressure along the limb 20 remains graduated as desired. An accurate amount of compressive pressure and properly graduated pressure helps ensure a desired level of therapy.

Similarly, the sleeve-wrap compression system 10 can advantageously provide the same change in compressive pressure across various degrees of stretching on limbs having different sizes. For example, the same compression garment would apply a different amount of compressive pressure to a limb having a 12-inch circumference than to a limb having a 7-inch circumference. However, in embodiments of the sleeve-wrap compression system 10, the stretch-compression characteristics of both the sleeve 12 and the wrap 14 are known and controlled. As a result, the change in compressive pressure from a light stretch (the first, about 30% stretch) of the wrap 14 to a firm stretch (the third, about 100% stretch) of the wrap 14 ranges about 20-30 mm Hg pressure (as illustrated by the example of some preferred embodiments) on any limb circumference to which the sleeve-wrap compression system 10 is applied. In other words, although the compressive pressure provided by a light stretch (the first, about 30% stretch) of the wrap 14 is different on a larger or smaller circumference limb, the change in compressive pressure provided by a firm stretch (the third, about 100% stretch) of the wrap can be about 20-30 mm Hg pressure greater in both the larger and smaller limbs.

The sleeve-wrap compression system 10 achieves a superior “working” compression profile compared to conventional compression systems. That is, the sleeve-wrap compression system 10 provides a consistent amount of compressive pressure over the course of clinical treatment of a wound. The individual features in the sleeve 12 and in the wrap 14, and the synergistic combination of those features, create a single compressive entity 10 that provides a controllable compression profile, particularly in response to a change in limb volume.

For example, as described herein, embodiments of the sleeve component 12 of the sleeve-wrap compression system 10 can include (1) an interior terry lining 30; (2) a reciprocated heel 32; (3) an open toe 34; (4) a contoured design; (5) stitch construction that permits horizontal stretch 40 with minimal vertical stretch 42; and (6) a low level of compressive pressure throughout the sleeve 12. Each of these aspects helps keep the sleeve 12 in a desired position on a limb 20 so that a stable compressive pressure can be maintained by the sleeve 12 and the overlying wrap 14. In addition, such features in the sleeve 12 provide a smooth dispersal of compression from the overlying wrap 14, thereby further enhancing control of compressive pressure onto the limb 20.

Embodiments of the wrap component 14 of the sleeve-wrap compression system 10 can include (1) defined amounts of compressive pressure correlated with various amounts of stretch; (2) a color change indicator system 50 that allows a user to readily determine a proper amount of stretch for controlling the amount of applied compressive pressure; and (3) stretch characteristics that provide long-stretch elastic compression similar to that in compression hosiery. Each of these aspects helps the sleeve-wrap compression system 10 maintain a stable, or consistent, interface pressure with a limb/wound over an extended wear/treatment period. In addition, friction co-efficiencies between the sleeve 12 and the wrap 14 help maintain the compression system 10 as a single compressive entity in proper position on a limb 20, which enhances control of compressive pressure on the limb 20.

The stretch characteristics of the wrap 14 allow the wrap 14 to provide a more elastic response to a change in limb volume, or girth, than responses by a stiffer system, such as a conventional cohesive wrap or four-layer wrap. Stiffness of a compression bandage, wrap, stocking, or other compression garment is measured in terms of slope value on an x/y (horizontal/vertical) axis. For purposes of illustration, stiffness slope value is the change in pressure produced by a 1 cm change in circumference of a limb 20. Change in limb circumference due to increase or decrease in limb volume affects the effective stretch of a compression garment. As increasing edema causes limb circumference to increase, stretch on the compression garment increases, and as decreasing edema causes limb circumference to decrease, stretch on the compression garment decreases. Stretch can be considered “stress” 60 on the garment, and is indicated on the x-axis in FIGS. 6-8. Thus, as stretch/stress 60 of a compression garment increases, the compressive pressure, or “strain” 62, applied by the garment increases. Likewise, as stretch/stress 60 of a compression garment decreases, the compressive pressure, or “strain” 62, applied by the garment decreases. Amount of compressive pressure/strain 62 is indicated on the y-axis in FIGS. 6-8.

As shown in FIG. 6, when a compression garment is stiff, it has a high slope value, that is, a steep stress/strain curve 64. In a stiff compression garment, a small increase in stretch/stress 60 (due to increase in limb circumference) results in a defined increase in actual compressive pressure 62. For example, in a stiff compression garment, a 1 cm increase in limb circumference may produce an increase in compressive pressure/strain 62 of 10 mm Hg. A conventional cohesive wrap, for example, exhibits such a high slope value, or stress/strain curve 64. FIG. 7 illustrates a stress/strain curve 64 for a moderately stiff compression device, that is, less stiff than a cohesive wrap yet not as elastic as a compression hosiery garment. In a moderately stiff compression device, a moderate increase in stretch/stress 60 (due to increase in limb circumference) results in the defined increase in actual compressive pressure 62. For example, in a moderately stiff compression device, an increase in compressive pressure/strain 62 of 10 mm Hg may be produced by a 2 cm increase in limb circumference. That is, in a moderately stiff compression garment, the same amount of increase in compressive pressure 62 as in a stiff compression garment is produced by a larger increase in limb circumference (a larger amount of stretch/strain 60). A conventional four-layer wrap, for example, exhibits such a moderate slope value, or stress/strain curve 64.

The comparative relationships between stretch/stress 60 and compressive pressure/strain 62 in FIGS. 6-7 illustrate that stiffness directly affects the ability to control a change in compressive pressure 62 in response to a change in circumference of a limb. Both stiff and moderately stiff compression garments have sufficiently high stress/strain curves 64 such that a small increase in edema/limb circumference can cause a relatively large increase in compressive pressure 62. The amount of applied compressive pressure 62 must be carefully controlled to ensure effective treatment of venous ulcers, as well as to prevent damage to tissue and/or arterial reflux from too large a pressure, particularly over time.

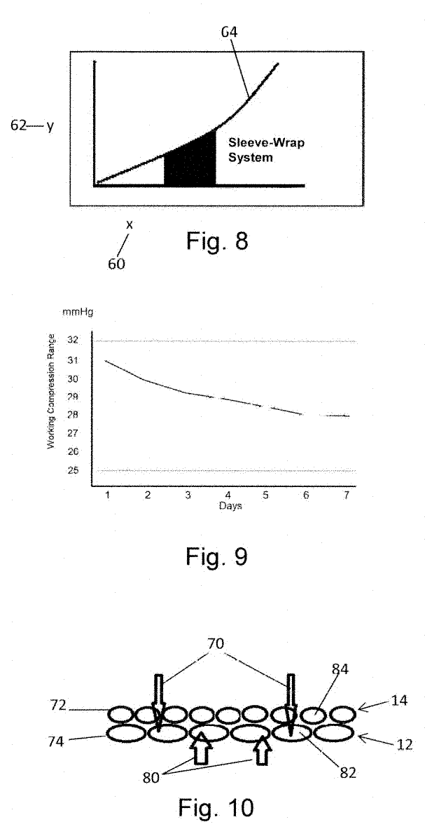

FIG. 8 illustrates the stress/strain relationship in the sleeve-wrap compression system 10. The sleeve-wrap compression system 10 exhibits less stiffness than a moderately stiff compression garment, such as a four-layer compression wrap, and has elasticity characteristics similar to that of a compression stocking. In the more elastic sleeve-wrap compression system 10, a larger change in stretch/stress 60 (due to a larger change in limb circumference) results in the defined change in actual compressive pressure 62. For example, in the relatively elastic sleeve-wrap compression system 10, an increase in compressive pressure/strain 60 of 10 mm Hg may be produced by a 5 cm increase in limb circumference. That is, in the relatively elastic sleeve-wrap compression system 10, the same amount of increase in compressive pressure 62 as in a stiff or moderately stiff compression garment is produced by an even larger increase in limb circumference (an even larger amount of stretch/strain 60). In other words, the relatively elastic sleeve-wrap compression system 10 exhibits a lower slope value, or stress/strain curve 64, than stiff or moderately stiff compression garments. Such a lower, more gradual stress/strain curve 64 is similar to that exhibited by a compression hosiery garment. As a result, the sleeve-wrap compression system 10 provides a more gradual change in applied compressive pressure 62 in response to a change in limb volume than stiff or moderately stiff compression garments, and particularly multi-layer compression wrap systems. Accordingly, the ability to provide a gradual change in applied compressive pressure 62 in response to a change in limb volume allows the sleeve-wrap compression system 10 to provide compressive pressure 62 within a defined, desired therapeutic range over time and with varying degrees of patient activity and rest. Maintaining compressive pressure 62 consistently within a desired therapeutic range during an extended course of treating venous ulcers can enhance healing outcomes.

In particular, recent research has shown that stiffness of a compression device affects venous ulcer healing rates. Stiff inelastic compression bandages and garments (which have a high stress/strain curve) rapidly lose therapeutic compression profiles as the volume of the limb decreases. Short-stretch bandages also have the disadvantageous tendency to lose a significant amount of pressure within the first few hours of application. For example, my testing showed that in one cohesive wrap applied on top of a second cohesive wrap about a cylinder, an initial 60 mm Hg compressive pressure dropped to about 20 mm Hg pressure after three hours. Stiff inelastic compression bandages can comprise tight, short-stretch bandages, such as one commercially available cohesive bandage under the name COBAN™ from 3M™ (3M Corporate Headquarters, 3M Center, St. Paul, Minn. 55144-1000), or semi-rigid zinc plaster bandages, such as one commercially available under the name Unna Boot from Medline Industries, Inc. (One Medline Place, Mundelein, Ill. 60060).

Elastic, or long-stretch, compression bandages and garments utilize the recoil force of elastic fibers to provide compression. As a result, elastic compression bandages and garments have advantages over inelastic bandages and garments by providing more consistent compression during changes in limb volume and during varying degrees of patient activity and by maintaining a constant interface pressure over a longer wear period.

Four-layer compression bandages combine aspects of both inelastic and elastic compression into one system. Such multi-layer systems include an absorbent pad layer, a crepe layer to hold the padding in place, a long-stretch bandage layer for providing compression, and a cohesive outer wrap. However, the stiffness of the cohesive outer wrap causes the predominant effect in such four-layer compression bandages to be similar to short-stretch bandages insofar as they do not provide significant compression during changes in limb volume. Over a 5-7 day wear cycle, four-layer compression bandages exhibit increasing slippage and substantial pressure loss (that is, less slippage and pressure loss than a purely inelastic bandage, but more than a purely elastic device). In addition, the wrapping procedure for a four-layer bandage is complex. An example of a such a four-layer compression bandage is one commercially available under the name PFOFORE® from Smith & Nephew Medical Ltd. (Hull HU3 2BN, England).

Moreover, with respect to healing of venous leg ulcers, O'Meara et al. have reported that multi-component systems (bandages or stockings) are more effective than single-component systems; that multi-component systems containing elastic, such as long-stretch elastic, are more effective than those composed mainly of inelastic, or short-stretch, constituents; and that two-component bandage systems perform as well as four-layer bandages.

Embodiments of the sleeve-wrap compression system 10 of the present invention comprise a multi-component system, preferably a two-layer system, comprising long-stretch elastic. As described, the sleeve-wrap compression system 10 exhibits a lower stress/strain curve 64 than stiff or moderately stiff conventional compression garments. Accordingly, the sleeve-wrap compression system 10 provides numerous advantages. For example, the sleeve-wrap compression system 10 provides the advantage of (1) easy application, in contrast to complex four-layer application procedures; (2) being maintained in a proper position on a patient's limb with minimal slippage; (3) consistent compressive pressure 62 during an extended wear period, for example, over 5-7 days; (4) a controlled, gradual change in applied compressive pressure 62 in response to a change in limb volume; and (5) consistent compressive pressure 62 during varying degrees of patient activity and rest. Each of these aspects of the sleeve-wrap compression system 10 allows the system to control applied compressive pressure 62 so as to maintain a consistent working compression profile. As a result, the sleeve-wrap compression system 10 can maintain an optimal, therapeutic level of compressive pressure 62 for the treatment of leg ulcers over time.

As described herein, design aspects of the sleeve component 12 of the sleeve-wrap compression system 10 and the interaction between the sleeve 12 and wrap 14, individually and together, help keep the two-layer system 10 in a desired position on a limb 20 so that a stable working compressive pressure can be maintained over time. Similarly, features of the wrap 14, including defined stretch/compressive pressure correlations, a stretch/compression color indication system 50, and stretch characteristics of the wrap 14, provide for maintenance of a consistent working compression profile. FIG. 9 illustrates that the sleeve-wrap system 10 maintains working compression within a desired range for seven days while the system 10 is being worn. As shown in FIG. 9, one exemplary embodiment of the sleeve-wrap compression system 10 that provides an initial working compression of about 31 mm Hg is able to maintain compressive pressure above about 28 mm Hg over a seven day period, which is within 90% of the initial working compression.

Embodiments of the sleeve-wrap compression system and/or method 10 of the present invention can comprise multiple compressive pressure layers. In preferred embodiments, the sleeve-wrap compression system 10 comprises a two-layer system in which a single compressive wrap layer 14 described herein is utilized in combination with the compressive sleeve layer 12. One advantage of such a two-layer compression system is that the sleeve 12 and the wrap 14 comprise features that combine to form a single compressive entity. When applied to a patient's limb 20, the inner sleeve 12 exerts a first compressive pressure that secures the inner sleeve 12 in a therapeutic position on the limb 20, and when applied by stretching over the inner sleeve 12, the outer wrap 14 exerts a second compressive pressure and frictionally engages the inner sleeve 12, thereby securing the compression system 10 as a single compressive entity in the therapeutic position on the limb. That two-layer, single-entity compression system 10 minimizes, if not eliminates, any potential of slippage and/or wrinkling between the two layers, 12, 14, respectively, thereby facilitating comfort for the patient and smooth dispersion of compression throughout the system 10. The two-layer, single-entity compression system 10 further provides consistent compressive pressure during an extended wear period and varying degrees of patient activity and rest, and a controlled, gradual change in applied compressive pressure in response to a change in limb volume. In these ways, the compression system 10 of the present invention can provide enhanced effectiveness in the treatment of venous leg ulcers and/or edematous conditions of body extremities.

In another embodiment of the present invention, another compressive wrap layer 14 can be applied on top of the first compressive wrap layer 14 to create a three-layer compression system. An embodiment having such a third layer continues to provide the benefits of the two-layer, single-entity compression system 10 over which the third layer is applied.

In yet other alternative embodiments, a different wrap can be utilized for the second and/or third layers. For example, in an alternative two-layer compression system 10, a cohesive wrap can be applied to the sleeve layer 12 in order to provide a more rigid pressure useful in certain therapeutic scenarios. Likewise, in an alternative three-layer compression system, a cohesive wrap can be applied as the third layer on top of the two-layer sleeve-wrap compression system 10. Other combinations of components of conventional compression systems with either the sleeve 12 and/or the wrap 14 of the present invention are also envisaged.

The sleeve-wrap compression system 10 may optionally include a wound dressing for covering and thus protecting an open wound, such as an ulcer, under the applied compression system.

The sleeve-wrap compression system 10 can comprise anti-microbial properties 70, as shown in FIG. 10. In some embodiments, the sleeve 12 comprises copper technology 74 on the interior of the sleeve 12. Anti-microbial copper technology 74 that can be integrated into fabric is commercially available from Cupron, Inc. (Richmond, Va.). Such copper technology 74 provides a broad spectrum of anti-bacterial, anti-viral, and anti-fungal activity, and can eliminate 99.9% of bacteria and fungi that cause odors. Thus, such anti-microbial copper technology 74 in the sleeve 12 effectively reduces odor from wound drainage, promotes wound healing, and protects skin around the wound.

In some embodiments, the wrap 14 comprises silver 72 integrated into the wrap 14. Silver 72 provides a broad spectrum of anti-bacterial, anti-viral, and anti-fungal activity 70. Accordingly, silver 72 in the wrap 14 can reduce odor from wound drainage wicked to the wrap layer 14 and help prevent infectious contamination of the exterior of the wrap 14.

FIG. 10 illustrates anti-microbial action 70 in the fibers of the wrap 14 and on the interior of the sleeve 12. As shown in FIG. 10, copper 74 comprised in the inner sleeve layer 12 and silver 72 comprised in the outer wrap layer 14 act together as a double barrier to reduce odor, prevent cross contamination from a wound, and promote wound healing. These anti-microbial properties 70 give the sleeve-wrap compression system 10 an advantage over conventional compression systems that may suppress odor but do not actively kill microbes in exudate from a wound.

In some preferred embodiments of the sleeve-wrap compression system 10, both the sleeve 12 and the wrap 14 comprise hydrophilic yarns 82, 84 that can wick 80 moisture/fluid from a wound and surrounding skin to the surface of the outer wrap 14. For example, the inner skin facing surface of the sleeve 12 can comprise knitted terry loops 30, which are hydrophilic 82 so as to absorb moisture/fluid from the underlying wound and skin surfaces and wick 80 it vertically outward away from those underlying surfaces. Once fluid/moisture is wicked 80 away from the surfaces of a patient's wound and/or skin by the hydrophilic yarns 82 in the sleeve 12, the fluid/moisture is wicked 80 through the sleeve layer 12 to the wrap layer 14, where hydrophilic yarns 84 continue to wick 80 the fluid/moisture to the surface of the wrap 14. FIG. 10 illustrate the presence of hydrophilic wicking fibers 82 in the sleeve 12 and vertical wicking 80 of moisture/exudate from a wound through the sleeve layer 12 and through the wrap layer 14 to the surface of the outer wrap layer 14. At the surface of the wrap 14, the fluid/moisture can evaporate into the air. Thus, vertical wicking 80 through two layers 12, 14 in the sleeve-wrap compression system 10 provides a system and method for managing draining wounds that need compressive pressure therapy. Wicking 80 moisture/exudate from a wound helps keep the wound drier, prevents wound maceration, and enhances skin comfort.

In some embodiments of the sleeve-wrap compression system 10, a secondary absorptive dressing, such as an ABD pad, can be placed on the outside of the wrap layer 14 to help absorb moisture/drainage wicked 80 away from a wound. Once soiled with drainage wicked 80 vertically outwardly from the wound by the sleeve 12 and wrap layers 14, the secondary dressing can be changed without having to change the sleeve-wrap compression system 10 or a primary dressing adjacent the wound.

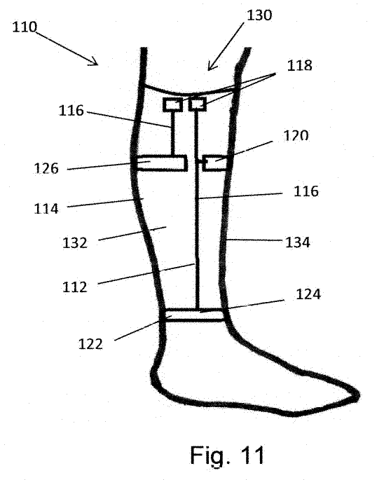

Some embodiments of the subject matter described herein include a compression and sensing system and method comprising a body monitoring system and method. FIGS. 11-20 illustrate such embodiments. In some embodiments, the body monitoring system 110 comprises a sensor configured to detect changes in one or more variables in a body. Various embodiments of the sensor can comprise electrical, mechanical, chemical, ultrasonic, acoustic, tactile, and/or other sensing mechanisms to monitor the intended variable(s). Embodiments of the body monitoring system 10 and/or method can be adapted to monitor variables in animate and/or inanimate bodies. Such variables include, for example, heartbeat, blood flow, pulse rate and quality, oxygenation, temperature, edema, body movements, and other physiological variables.

As shown in FIG. 11, the body monitoring system 110 can comprise an electrically conductive yarn 112 knitted into a fabric or garment 114 as a transmission circuit 116. The transmission circuit 116 provides a pathway for transmitting electrical signals representing a value of a monitored variable from a sensor located on the fabric/garment 114 to a display unit 118 where the variable value can be displayed. The sensor can comprise various forms and functionalities. For example, as illustrated in FIG. 11, the sensor can comprise the electrically conductive yarn 112 knitted into the fabric or garment 114 as a sensing circuit 120. In another embodiment, the sensor can be integrated into a cuff 122 that is knitted about the circumference of the tubular garment 114 (cuff sensor 124). In another embodiment, the sensor can be integrated into a pocket 126 that is knitted in a discrete area of the garment 114. The transmission circuit 116, sensing circuit 120, cuff sensor 124, pocket 126 adapted to contain a sensor, and display unit 118 are described in detail below. Other embodiments of the sensor and other aspects of the present invention are also described below.

In one illustrative embodiment, the body monitoring system 110 and/or method can comprise a system and/or method for monitoring compression in a body. Reference is made throughout this description to a body compression monitoring system 130 and/or method for purposes of illustration only. The inventive features of the present invention apply to systems and/or methods for monitoring a variety of variables other than compression and in different kinds of bodies.

As shown in FIG. 11, one embodiment of such a body compression monitoring system 130 can comprise a compressive pressure garment, wrap, bandage, or device 132 (collectively “compressive pressure device” or “device”) that incorporates into the system 130 an ability to monitor compressive pressure applied by the device 132 on a body. For purposes of illustration, the compressive pressure device 132 in FIG. 11 is configured to be worn on a person's lower limb 134. The body compression monitoring system 130 and/or method provides a mechanism for easily and accurately determining an actual amount of compressive pressure applied to an anatomical area by the compressive pressure device 132. The actual applied compressive pressure can be measured in mm Hg, for example. The body compression monitoring system and/or method can further comprise the display unit 118, or mechanism for displaying measurements of the applied compressive pressure.

Various types of sensors configured to measure applied compressive pressure can be utilized in the body compression monitoring system 130 and/or method. A particular embodiment of such a body compression monitoring system 130 can include a single type of sensor or a combination of different types of sensors.

In some embodiments, the body monitoring system 110 can comprise a pathway from the sensor to the electronic display unit 118 where the value of a measured variable can be displayed. The pathway can have various dimensions and take various paths from the sensor to the display unit 118. The pathway can comprise a vertical path along the longitudinal axis of a wearable device 140, for example, along a wale 136 or a selected number of adjacent wales 136 in the knitted compressive pressure device/garment 132. For example, the pathway can extend from a sensor in the compressive pressure device 132, such as about an ankle, vertically to the display unit 118 at the top of the device 132. Measurements of applied compressive pressure by the sensor can be transmitted to the display unit 118 in the form of an electrical signal. Accordingly, the pathway can be referred to as a transmission circuit 116. Examples of vertical pathway transmission circuits 116 are shown in FIGS. 11, 12, 13, and 19.

The transmission circuit 116 can comprise electrically conductive yarn(s) 112. For example, the transmission circuit yarn 112 can be an electrically conductive silver yarn or a yarn coated with silver. Various commercially available silver yarns are useful in embodiments of the present invention. One preferred silver yarn is X-STATIC®, commercially available from Noble Biomaterials, Inc. (300 Palm Street, Scranton, Pa. 18505). The X-STATIC® silver yarn comprises 99.9% pure elemental silver and is highly electrically conductive, lightweight, flexible, stretchable, washable, and durable. In addition, the X-STATIC® silver yarn is a broad spectrum antimicrobial and odor eliminator useful in the care of wounds such as dermal ulcers.

The transmission circuit pathway 116 can be integrally knit into the wearable device 140 while the device 140 is being knit. During the process of knitting a tubular wearable device 140 on a circular knitting machine, yarns being knit for the device 140 are cut at a predetermined location about the device circumference. An electrically conductive yarn 112 is then picked up and dropped in for a selected number of cycles, for example, four cycles. After being knit for the selected number of cycles, the electrically conductive yarn 112 is dropped, and the yarn for knitting the device 140 is picked back up to continue knitting around the device circumference. These steps are repeated so as to construct the vertical transmission circuit 116, or stripe. In some embodiments, the transmission pathway circuit 116 comprising the knitted electrically conductive yarn 112 can be knit on a flat bed knitting machine.

In another embodiment, the wearable device 140 can comprise polyester yarn, and the transmission pathway (circuit) 116 can comprise nylon yarn. Once the wearable polyester device 140 having a nylon yarn transmission pathway 116 is fabricated, the entire device fabric can be coated with silver or a silver composition. Because silver adheres to nylon but not to polyester, only the transmission pathway 116 is coated with the silver or silver composition. As a result, the nylon pathway is provided with an electrically conductive material to create the transmission circuit 116. To further assure that the silver-coated nylon stitches in the transmission circuit 116 are sufficiently packed together to provide a continuous circuit, the wearable device 140 can be heated. Heating the device 140 a particular amount shrinks the nylon yarn so as to further pack the nylon-silver yarns along the transmission circuit 116 for enhanced conductivity.

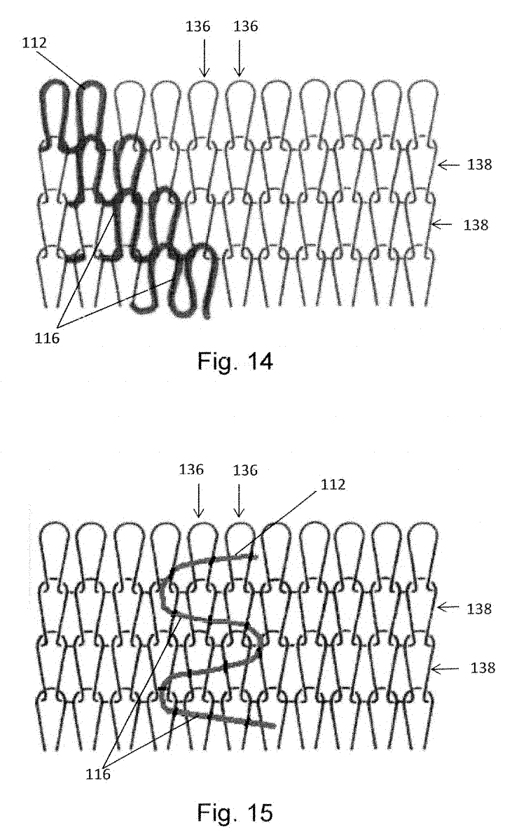

In embodiments of the body monitoring system 110 and/or method, transmission circuits comprising electrically conductive yarns 112 can be knit in fabrics in any direction. That is, electrically conductive yarn circuits 112 can be knit vertically, horizontally, or at angles in a fabric. The direction and specific path of the transmission circuit 116 can be determined by the selection of stitch pattern and conductive yarn. An angled transmission pathway circuit 116 can be knit utilizing either cut yarns or a continuous yarn. To achieve an angled transmission circuit utilizing cut yarns, the electrically conductive yarn 112 can be knit in a wale 136 offset from a previous wale 136 in successive courses 138 as the fabric is knitted in the vertical direction. FIG. 14 shows an example of an angled transmission pathway circuit 116 having a cut electrically conductive yarn 112. Such angled circuits 116 facilitate the use of sensors in various locations on a body, for example, about anatomical curvatures.

A horizontal transmission circuit 116 can be achieved by knitting the electrically conductive yarn 112 horizontally, or laterally, in a fabric along one or more courses 138. Alternatively, a continuous electrically conductive yarn 112 can be “laid in” a knitted fabric structure, for example, along one or more courses 138, to provide a horizontal transmission circuit 116. In certain embodiments, a continuous electrically conductive yarn 112 can be “laid in” a fabric structure so as to have changing directions to provide a transmission circuit 116 along a particular desired pathway. For example, FIG. 15 shows the electrically conductive yarn 112 “laid in” a fabric structure in a serpentine manner to provide the transmission circuit 116 at particular locations in the fabric. Providing the transmission circuit 116 at particular locations in this manner allows placement of sensors at desired locations in the fabric. The continuous electrically conductive yarn 112 can also be “laid in” a knitted fabric structure to provide an angled transmission pathway circuit 116.

In one aspect of the present invention, the electrically conductive transmission pathway, or circuit, 116 can be knit into a stretch fabric, that is, fabric having elasticity. Reliability of signal transmission along the pathway 116 depends, at least in part, on the continuity of the circuit 116. Circuit continuity relates primarily to yarn contact along the pathway 116. In some embodiments, circuit continuity can be enhanced by increasing yarn contact with a knit construction that packs stitch loops compactly together and/or shrinking a nylon-based pathway yarn by heating. In embodiments of an elastic fabric comprising the electrically conductive transmission pathway 116, circuit continuity can be further enhanced by limiting stretch in the direction of the pathway 116. In this way, reliable contact for conductivity can be maintained between stitches of the electrically conductive yarn 112 along the pathway 116.

For example, in embodiments of such an elastic fabric having the transmission circuit pathway 116 knit in the vertical direction, vertical stretch in the fabric can be limited. The limit of vertical stretch desirable in a stretch fabric depends on whether the electrically conductive yarn 112 in the transmission pathway 116 is knit in a cut manner or in a continuous, uncut manner.

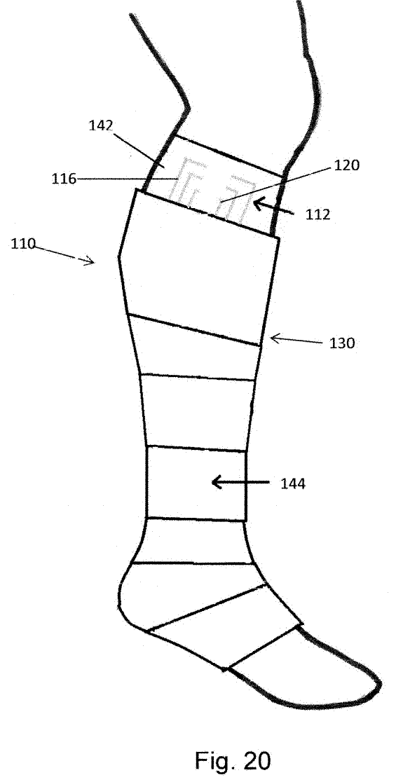

In embodiments in which the electrically conductive yarn 112 is knit in a cut manner, stretch in the direction of the transmission pathway 116 is preferably limited to about 5-10% beyond the unstretched, or resting, dimension of the fabric in the pathway direction. For example, in a rectangular, or elongated, compressive pressure wrap 144 (as shown in FIG. 20) having the transmission pathway 116 knit in the vertical direction along the length of the wrap 144, vertical (or longitudinal) stretch is preferably limited to about 5-10% beyond the unstretched length of the wrap 144. In “cut yarn” knitting on a circular knitting machine, the electrically conductive yarn 112 is brought up in one or more needles to the tuck height where the yarn 112 is cut. The cut electrically conductive yarns 112 in adjacent wales 136 are tightly knit, or packed together, so as to provide continuous contact between the cut yarns 112 to form the transmission circuit 116 in the vertical direction. It was further discovered that washing a fabric having a cut yarn transmission pathway 116 causes the tails of the cut yarns 112 to draw inward toward adjacent cut yarns 112 to improve electrical conductivity along the pathway 116. In embodiments in which the electrically conductive yarn 112 is knit in a continuous, uncut manner, the amount of stretch in the direction of the transmission pathway 116 permissible to maintain sufficient electrical conductivity depends on the type of conductive yarn 112. For example, when the electrically conductive yarn 112 is a conductive stretch nylon, stretch in the direction of the transmission pathway 116 is preferably limited to about 10-20% beyond the unstretched, or resting, dimension of the fabric in the pathway direction. Additional permissible stretch can be achieved by utilizing yarn having a higher stretch modulus. For example, when the electrically conductive yarn 112 is a 70 denier spandex yarn, single or double covered with a conductive nylon yarn, stretch in the direction of the transmission pathway 116 can be about 50-100% beyond the unstretched dimension of the fabric in the pathway direction without diminishing conductivity sufficient for signal transmission.

Although stretch in the direction of the knitted transmission pathway 116 is preferably limited, embodiments of such elastic fabrics can have substantial stretch in the direction opposite the direction of the transmission pathway 116 without affecting transmission of an electrical current signal along the pathway 116. As discussed, preferred limitations of stretch depend on the direction of the transmission pathway 116 and the construction of the pathway circuit 116. For example, in an elastic fabric having a pathway 116 knit in the vertical direction, the fabric can be stretched in the horizontal direction without affecting transmission of an electrical current signal along the vertical pathway 116.

The vertical pathway transmission circuit 116 can be knit using various knit patterns. In a preferred embodiment, the vertical pathway transmission circuit 116 is knit in a rib pattern. In a rib stitch pattern, wales 136 are alternated between the face of the fabric and the back of the fabric. The rib pattern can be two, threes, or four needles (or wales 136) wide, for example. In the transmission circuit 116 knit in a rib pattern, silver can be plated on one side of the rib, preferably the back side of the rib. The rib pattern can be either an elastic or a nonelastic rib pattern, which can be programmed into the knitting machine.

Conductivity properties in the knitted transmission circuit 116 and in the knitted sensing circuit 120 can vary depending on a number of factors, including the type of electrically conductive yarn 112, yarn size (denier), yarn construction, amount of yarn in a given area (yarn/fabric density), and stitch pattern. That is, such factors can be balanced in a fabric structure to achieve conductivity in the circuit 116, 120 suitable for reliably sensing and transmitting signals. For example, an electrically conductive silver yarn has different conductivity properties than an electrically conductive stainless steel yarn. A knitted-in circuit 116, 120 comprising a yarn having a first denier has different conductivity properties than a knitted-in circuit comprising a yarn having a second, different denier. Yarn sizes suitable for reliable signal transmission conductivity in some sensor applications include yarns in the range of about 70 denier to about denier. Reliable signal transmission conductivity may also be achieved in more sheer fabrics having smaller denier yarns. As an example, a single 70 denier silver yarn provides for transmission of a reliable electrical signal in some sensor applications/embodiments. In other applications/embodiments, two 70 denier silver yarns twisted together to form a 140 total denier yarn provides for transmission of a reliable electrical signal. In still other embodiments, the electrically conductive yarn 112 can be a covered stretch yarn.

A larger amount, or density, of yarn 112 in a knitted-in circuit generally exhibits greater conductivity than a smaller density of yarn 112. A knitted-in circuit 116, 120 comprising a standard single jersey stitch pattern has different conductivity properties than a knitted-in circuit 116, 120 comprising a different stitch pattern. Likewise, different selections of a rib pattern may affect conductivity in the knitted-in circuits 116, 120. For example, a 2×2 rib selection may have different conductivity than a 1×1 rib selection. Thus, by altering the yarn type, size, amount, and pattern in the knitted circuits 116, 120, the flow of electrical signals can be controlled. As a result, the type of variables being monitored and the manner in which those variables are monitored can be controlled.

In addition, various combinations of such conductivity factors can be utilized in different sections of the garment 114. In this way, the flow of electrical signals/current can be controlled as desired for monitoring multiple variables in the same garment 114. Similarly, the dimensions of the knitted-in circuits 116, 120 can be varied by programming the knitting machine to knit different widths, lengths, and/or shapes of the circuits 116, 120. Circuits 116, 120 having different dimensions in the fabric/garment 114 can have different conductivity properties that can be utilized for different purposes in the same fabric/garment 114.

During the process of knitting the body monitoring system 110, such as in the process of knitting the compressive pressure device 132, the electrically conductive yarns 112 knit in the vertical transmission circuit 116 are preferably “packed” together vertically. That is, the electrically conductive yarns 112 are knit tightly so that the stitch loops in adjacent courses 138 along a particular wale 136 are compacted together. In this way, the electrically conductive yarns 112 in adjacent courses 138 have sufficient contact to provide a continuous circuit. Such a continuous circuit allows transmission of an electrical signal representing a compressive pressure measurement from a sensor to another location, such as the electronic display unit 118.

In some conventional tubular compressive pressure garments, yarn stitches in the upper portion of the garment are knit more loosely than in the rest of the garment to provide a more tailored fit about a larger upper part of the limb on which it is to be worn. However, in the compressive pressure device 132 having the vertical transmission circuit 116, yarns 112 in the circuit 116 are preferably knit tightly in the entire extent of the circuit 116 to provide sufficient yarn contact throughout the circuit 116 for reliable signal transmission.

The transmission circuit 116 is connected to the sensor with an interface appropriate for the type of sensor. For example, a different type of interface can be utilized to connect the transmission circuit 116 for each of the knitted cuff sensor 124, a stand-alone electrically conductive yarn sensor, a separate electro-mechanical, capacitance, or piezoelectric sensor housed within the cuff 122 or pocket 126, or other sensor. In each instance, the transmission circuit connection with the sensor is configured to allow transmission of an electrical signal representative of a value of a sensed variable to the display unit 118 where the value of a sensed variable can be displayed.

The number of transmission circuits 116 in the wearable device 140 can vary, depending on the number of sensors in the device 140 from which measurements of a variable are to be transmitted. Transmission circuits 116 can be placed at different locations about the wearable device 140 as desired. For example, three vertical transmission pathways 116 can be placed on two different sides of the tubular device 140, one circuit 116 each for a sensor on the lateral aspect and the medial aspect of the instep, ankle, and calf.

While the knitted-in transmission circuit 116 is a preferred mechanism for transmitting a measure, or value, of a variable, such as an amount of applied compressive pressure, to the display unit 118, other mechanisms are contemplated. For example, an electrically conductive wire, such as a copper wire, can be utilized to transmit signals representing measurements of the variable from the sensor to the display unit 118. In such an embodiment, the copper wire can be integrated into the fabric of the wearable device 140, either by knitting the wire in the fabric 140 or by laying in the wire during construction of the device 140. Alternatively, such a wire can be attached externally to the wearable device 140.

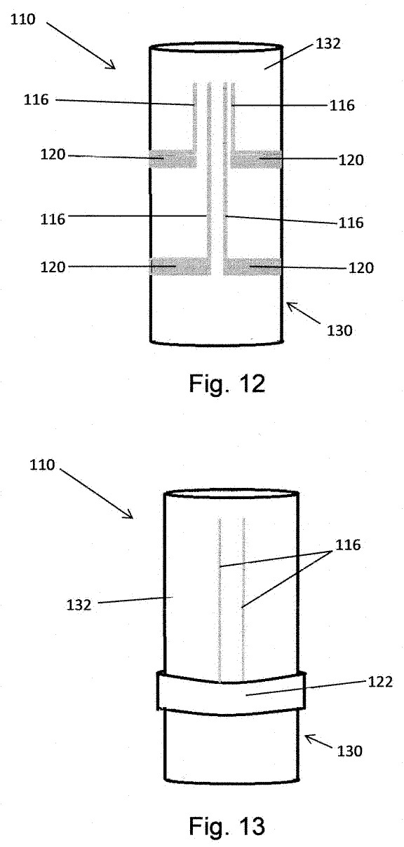

In some embodiments of the body monitoring system 110 and/or method, the sensor can be a knitted-in sensor circuit 120. The knitted-in sensor circuit 120 can be constructed using electrically conductive yarn 112 in a manner similar to the knitted-in transmission circuit 116. An advantage of the knitted-in sensor circuit 120 is that it can be knit to have various shapes and/or dimensions and placed in desired locations throughout the wearable device 140. Configuration and positioning of the knitted-in sensor circuit 120 can readily be accomplished by programming a knitting machine. One preferred shape of the knitted-in sensor circuit 140 is a rectangle, positioned horizontally about a tubular wearable device 140, such as the compressive pressure garment 132, as shown in FIGS. 11 and 12. The knitted-in sensor circuit 120 can be adapted to measure one or more variables, such as applied compressive pressure, at various points throughout the sensor dimension. Such a sensor having a horizontal orientation about a wearer's limb can thus provide measurements of the variable(s), such as applied compressive pressure, about an entire anatomical plane.

The sensor circuit 120 can be knit into the fabric of the wearable device 140. In one embodiment, the wearable device can comprise a compression sleeve 142, as shown in FIG. 20. In this way, when the sleeve 142 is worn without an overlying application, such as a compression wrap 144, the compressive pressure applied by the sleeve 142 can be measured. In addition, when the wrap 144 or other compressive pressure device is applied on top of the sleeve 142, the cumulative compressive pressure of the inner sleeve 142 and the outer wrap 144 or device can be measured.

In some embodiments, the knitted-in circuit can be a circuit that only transmits an electrical signal. In other embodiments, the knitted-in circuit can be a circuit that only senses a variable in the area of a body to which the wearable device 140 is applied. In yet other embodiments, the knitted-in circuit can be both the sensing circuit 120 and the transmission circuit 116.

In another aspect of the present invention, certain knitted-in circuits 116, 120 may be configured to transmit power from a power source to a device within or on a fabric, garment, or bandage. Power transmitted from an external power source to a location in the fabric/garment 114 can be utilized for various purposes. Such purposes can include, for example, direct electrical stimulation therapy, heating the fabric, or powering a device, such as a transcutaneous electrical nerve stimulator unit or a miniature air pump.

In some embodiments, the wearable device 140 can comprise the electrically conductive transmission pathway 116 constructed so as to allow electrical transmission in both directions along the pathway 116. In such embodiments in which an electrical current can travel in both directions, one part of the circuit 116 can be configured to transmit an electrical signal representing the value of a sensed variable from a sensing area on the body to the external electronic display unit 118, and another part of the circuit 116 can be configured to transmit an electrical current, such as powerable current, from a first location in the wearable device 140 to second location in the device 140 or from a location separate from the device 140 to a desired location in the device 140.

One sensor comprises the cuff 122 integrally knit into the fabric of the wearable garment or device 140, such as the compressive pressure device 132 shown in FIGS. 11 and 13. The cuff sensor 124 comprises electrically conductive yarns 112 capable of sensing a variable, such as the amount of compressive pressure being applied. In some embodiments, the knitted cuff sensor 124 is constructed to have three knitted fabric layers—a first layer comprising a base fabric layer of the wearable device 140; a second layer comprising an inside layer of the cuff 122; and a third layer comprising an outside layer of the cuff 122. That is, the cuff 122 can be constructed to overlie the first, device layer. The second, inside layer of the cuff 122 lies adjacent the first, device layer. The cuff 122 can have a length such that it can be folded over onto itself, such that the third, outside layer of the cuff 122 is adjacent the second, inside cuff layer.

In one embodiment, the knitted cuff sensor 124 comprises a capacitance type sensor. In one knitted cuff, capacitance type sensor, the first, base layer of the wearable device 1 40 comprises an inner electrically conductive yarn 112. The second, inside layer of the cuff 122 comprises a semi-conductive yarn. And, the third, outside layer of the cuff 122 comprises an outer electrically conductive yarn 112. With an electric current running through the inner and outer conductive yarns 112, the separation between the first and third fabric layers can be measured to provide a capacitance value for the measurement area. Such a capacitance value can be correlated to, for example, an amount of compressive pressure being applied by the wearable device 140. A change in capacitance value can thus be correlated with an amount of change in applied compressive pressure.

The electrically conductive yarn(s) 112 in both the first, base layer of the device 1 40 and in the third, outer cuff layer can comprise yarn such as silver yarn or stainless steel yarn. One preferred silver yarn for the knitted cuff sensor is X-STATIC®, commercially available from Noble Biomaterials, Inc. Alternatively, the first, base layer of the device 140 and the third, outer cuff layer can comprise nylon and polyester yarns. The layers can be constructed so that the nylon yarns are in a particular pattern configured for sensing an area of compressive pressure. A conductive silver composition can be applied to the first and third layers, whereby the silver composition adheres to the nylon but not to the polyester. In this way, the silver-coated nylon yarns can function to carry an electrical current and act as capacitance-based compression-sensing bars, or nodes.

The knitted cuff sensor 124 can be constructed so that the range, or spread, of electrical conductivity (sensitivity) in a sensing area is broad enough to reliably detect differences in a variable, such as compression, represented by an electrical signal. For example, in some embodiments, the range of electrical sensitivity can be between about 5-15 kOhms. In other embodiments, electrical conductivity/sensitivity can comprise other ranges, depending on the variable being sensed. In testing, it was discovered that some silver yarns are too conductive to transmit electrical signals in such a desired sensing range. The preferred X-STATIC® silver yarn provides a range of electrical conductivity/sensitivity that allows sensing variables in embodiments of the present invention.

In another embodiment of a knitted cuff, capacitance type sensor, each of the inside layer and the outside layer of the cuff 122 comprises the electrically conductive yarn 112. An electrically regulating dielectric insulator material can be inserted between the two layers of the cuff 122. In this configuration, capacitance between the two electrically conductive layers of the cuff 122 can be measured as a function of compressive pressure applied by the compressive pressure device 132. That is, as the limb 134 on which the compressive pressure garment or device 132 is being worn swells or otherwise changes shape, increasing pressure at the interface between the limb 134 and the garment/device 132 will likewise be applied to the interface of the garment/device 132 with the knitted cuff 122. In this way, the cuff sensor 124 can sense changing pressure applied to the underlying limb 134.

In another embodiment, the knitted cuff sensor 124 comprises a piezoelectric type sensor. A piezoelectric pressure sensor measures changes in pressure by converting those changes to an electrical charge. In one knitted cuff, piezoelectric type sensor, the first, base layer of the wearable device 140 comprises a non-conductive plate portion integrated with or attached to the layer. The second, inside layer of the cuff 122 comprises a conductive material, for example, a copper wire knit into the fabric of the second layer. And, the third, outside layer of the cuff 122 comprises a non-conductive plate portion integrated with or attached to that layer. The non-conductive plate portions can be a plastic material, for example. As the two non-conductive plate portions move in relation to each in response to changing compressive pressure exerted by the device 140, the force field between the plates changes. The change in pressure between the plates can be measured as a change in electrical charge carried along the copper wire.

In another embodiment, the knitted cuff sensor 124 comprises a piezoresistive type sensor. In such a sensor 124, the first, base layer of the wearable device 140 comprises an inner electrically conductive yarn 112. The second, inside layer of the cuff 122 comprises a piezoresistive semi-conductive polymer. The piezoresistive material comprises an electrical resistivity that varies inversely with pressure exerted on the material. And, the third, outside layer of the cuff 122 comprises an outer electrically conductive yarn 112. The inner and outer electrically conductive yarn(s) 112 in the first and third layers can comprise any electrically conductive yarn, and preferably is a silver yarn. In such a piezoresistive sensor, a change in compressive pressure applied by the device 140 causes a change in resistance between the two layers (first and third layers) comprising electrically conductive yarns 112. The change in resistance can be converted to an electrical signal representative of a correlated amount of applied compressive pressure.

Embodiments of the body monitoring system 110 can have one or more cuff sensors 124, as shown in FIGS. 11 and 14, knit into the wearable device 140. The cuff(s) 122 can be knit at location(s) along, for example, the compressive pressure garment/device 132 desired for measuring applied compressive pressure at such location(s). For example, cuffs 122 can be knit at the calf, ankle, and/or instep in the compressive pressure device 132 designed for the lower limb 134. Embodiments of the body monitoring system 110 having the knitted cuff sensor 124 can be manufactured all in one step, for example, on a circular knitting machine. That is, the circumferential cuff 122 can be integrally knit while the wearable device is being knit. In a one embodiment, the compressive pressure device 132 and cuff 122 can be knit with a Lonati Model GL615 electropneumatic single cylinder circular knitting machine. This machine has a 168-needle cylinder containing 3¾ inch medium butt and short butt needles typically used for knitting socks. The machine includes a single main feed with eight yarn finger selections, one elastic selection at the main feed, and five pattern feeds. One elastic station has two elastic selections.

During knitting of the compressive pressure garment 132, the cuff 122 can be knit at a desired location. Beginning with a circular knitting motion, the cuff 122 can be knit by loading the needles using a 1×1 selection at the main feed for one revolution of the needle cylinder, with a yarn delivered by one of the yarn fingers at the main feed. In the second revolution, all needles come up to knit height for one revolution to lock the stitches onto the needles. In the third revolution, the cylinder needles change to a 1×1 selection opposite from selection in the first revolution, and the dial jacks are loaded with yarn by moving out between the cylinder needles that are down for one revolution.

The knitting machine can be programmed to operate as in the third revolution for a set number of courses 138 to obtain a desired length for the cuff 122. After a set number of courses 138 for the cuff 122 has been knit, dial cams for controlling the dial jacks are activated. This causes the dial jacks to move out over the cylinder needles so that yarn being held by the dial jacks is transferred back onto the cylinder needles to complete the cuff 122. In this manner, the knitted cuff sensor 124 can be integrally knit into the compressive pressure device 132. Various yarns and stitch patterns can be knitted into the garment device 132 and cuff 122 sections to create various types of sensors as described herein. In certain embodiments, different yarns and stitch patterns can be used for each of the inside layer and the outside layer of the cuff 122.

In some embodiments of the body monitoring system 110 and/or method, the sensor can comprise the electrically conductive yarn 112 knit into the wearable device. For example, the compressive pressure device 132 can be knit such that the electrically conductive yarn(s) 112 are positioned at desired locations for measuring compressive pressure. An amount of applied compressive pressure can be sensed by the yarn(s) 112 and converted to an electrical signal representative of an amount of pressure. In one such embodiment, the electrically conductive sensor yarn(s) 112 can be knit into an inner surface of the fabric of the compressive pressure device 132 so that those yarns 112 are in contact with an underlying body. In another embodiment, the cuff 122 can comprise the electrically conductive yarn circuit 120 configured to sense one or more variables in a body. The sensor circuit 120 in the cuff 122 can be connected to the knitted transmission pathway circuit 116.

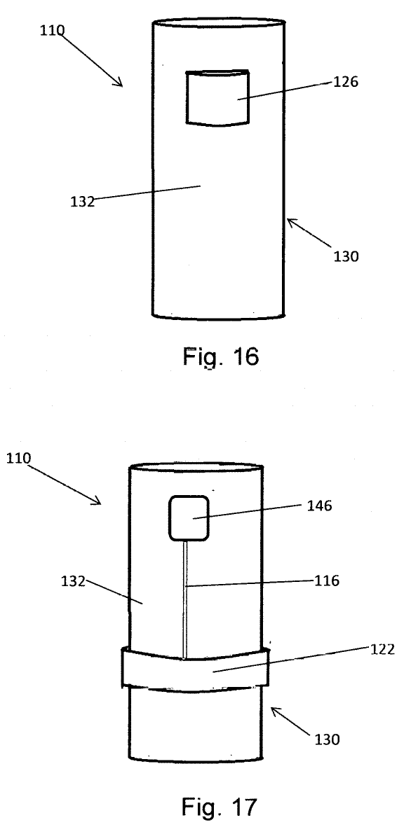

Embodiments of the body monitoring system 110 can have one or more pockets 126, as shown for example in FIGS. 11 and 16, knit into the wearable device 1 40. A separate sensor can be placed into, or housed in, the pocket 126. One advantage of the body monitoring system 110 in which a separate sensor is placed in the pocket 126 is that stretching of other layers of the wearable device 140 has minimal effect, or no effect, on the measurement of the variable(s) at the sensor location. The pocket(s) 126 can be knit at location(s) along a compressive pressure garment/device 132 desired for measuring applied compressive pressure at such location(s). For example, pockets 126 can be knit at the calf, ankle, and/or instep in the compressive pressure device 132 designed for the lower limb 134. Accordingly, actual compressive pressure at each of the locations at which a sensor is located can be accurately measured.

Embodiments of the body monitoring system 110 having the knitted pocket 126 can be manufactured all in one step, for example, on a circular knitting machine. That is, the pocket 126 can be integrally knit while the compressive pressure garment/device 132 is being knit. For example, using the Lonati circular knitting machine described herein, the pocket 126 can be knit at a desired location during knitting of the compressive pressure garment 132. To construct a knitted-in pocket 126, the needle cylinder moves from a circular motion into a reciprocated motion using medium butt needles. Needle lifters are used to raise the needles one at a time, one in each direction of reciprocation, and needle droppers are used to lower the raised needles down to knitting height out of action. The machine then reciprocates knitting on the medium butt needles only for a set number of courses to form the pocket 126. By holding the needle lifters and needle droppers out of action and open on each side, a seamless pocket 126 can be knitted. In this manner, the pocket 126 can be knitted either on the inside surface or on the outside surface of the compressive pressure device 132.

A compressive pressure sensor can be placed inside the pocket 126 for monitoring compressive pressure applied at the pocket location. In addition to sensors, various other devices such as, pumps, wireless transmitters, batteries, and/or other components related to a compressive pressure device 132 can be placed inside the pocket 126. One advantage of housing a device inside the pocket 26 is that the sensor or component is securely maintained in a desired position, while the sensor or component does not touch the skin of the wearer.

In similar fashion as the pocket 126, the cuff 122 integrally knit into the compressive pressure device 132 according to a method of the present invention can serve to house a separate compressive pressure sensor or other device. When the cuff 122 is utilized to hold a separate compressive pressure sensor in position in a desired location, the cuff 122 is preferably a non-sensing cuff. That is, in this application, the cuff 122 is knit without electrically conductive yarns 112.

In some embodiments of the body monitoring system 110 and/or method, the sensor can be an electro-mechanical sensor. The separate electro-mechanical sensor can be placed into, or housed in, the pocket 126 and/or the cuff 122 knit into the wearable device 140. Accordingly, a value of a variable at each of the locations at which the electro-mechanical sensor is located can be accurately measured.

One electro-mechanical sensor useful in a body monitoring system 110 and/or method is a flat force sensor. For example, the flat force sensor can be a force-sensing resistor (FSR) that exhibits a decrease in resistance when there is an increase in the force applied to the resistor. Thus, the resistor-sensor is able to detect force or pressure, including compressive pressure applied by the compressive pressure garment/device 132. In one embodiment, the resistor-sensor can comprise a polymer thick film (PTF) optimal for sensing an applied force ranging from a few dozen grams to over 10 kg. The resistor-sensor is preferably an elongated strip, approximately ½-¾ inch wide, and can have an active sensing area that is about ¼ inch wide. The resistor-sensor strip is desirably thin (for example, about 0.025 inch) and flexible, yet does not appreciably compress when pressure is applied. Such a force-sensing resistor is commercially available from Interlink Electronics, 546 Flynn Road, Camarillo, Calif., 93012 (www.interlinkelectronics.com). As a result, the force-sensing resistor sensor can be inserted flat or with only a slight curve within the cuff 122 or pocket 126 on the compressive pressure device 132 so as to maintain accuracy of pressure measurements.

In some embodiments of the body monitoring system 110 and/or method, the sensor can be a capacitance sensor. The separate capacitance sensor can be placed into, or housed in, the pocket 126 and/or the cuff 122 knit into the wearable device 140. Accordingly, a value of a variable at each of the locations at which the capacitance sensor is located can be accurately measured.

A capacitance sensor typically comprises two parallel plate conductors and an insulator between the two plates. Capacitance is directly proportional to the surface area of the parallel plates and inversely proportional to the separation distance between the plates or the displacement of one plate relative to the other plate. Capacitance can be calculated as the area of overlap of the two plates multiplied by a dielectric constant (relative static permittivity) and an electric constant, divided by the separation between the plates. Thus, a particular separation between two plates can be measured as a capacitance value for the measurement area. Such a capacitance value can be correlated to an amount of compressive pressure being applied by the compressive pressure device 32. A change in capacitance value can thus be correlated with an amount of change in applied compressive pressure.

In some embodiments of the body monitoring system 110 and/or method, the sensor can be a piezoelectric sensor. A piezoelectric pressure sensor measures changes in pressure by converting those displacement changes to an electrical charge. The separate piezoelectric sensor can be placed into, or housed in, the pocket 126 and/or the cuff 122 knit into the wearable device 140. Accordingly, a value of a variable at each of the locations at which the piezoelectric sensor is located can be accurately measured.

As described herein, the body monitoring system 110 and/or method can comprise the cuff 122 integrally knit with the compressive pressure device 132 in such a manner that the cuff 122 itself comprises the sensor. Alternatively, the cuff 122 and/or the pocket 126 can be knit into the wearable device 140 and configured to hold a separate sensor inside the pocket 126 or cuff 122. The separate sensor can be an electro-mechanical sensor, a capacitance sensor, or a piezoelectric sensor. Similarly, the non-sensing cuff 122 and/or pocket 126 can be adapted to house other devices and/or components related to a particular wearable device 140. For example, in one particular embodiment, the knitted-in cuff 122 can be constructed to hold an adjustable air bladder, as shown in FIG. 17. The air bladder housed in the knitted-in cuff 122 can be connected to an air pump 146 via the transmission circuit 116.

In some embodiments, the sensor can be attached to the wearable device 140 using a hook-and-loop type fastening system. For example, a surface of the wearable device 140 can comprise one portion 154 of a hook-and-loop type fastener that is engagable with a mating portion 156 of such a fastener. The sensor can be secured to a strip of material comprising the mating portion 156 of the fastener. By attaching the sensor-containing strip of the mating portion 156 to the hook-and-loop fastening enabled wearable device 140, the sensor can be reliably secured to the device 140.

The wearable device 140 using a hook-and-loop type fastening system can include an engagable portion 154 of the fastening system over the entire surface of the device. In this way, a mating portion 156 of the fastener having an attached sensor can be positioned for measuring the variable(s) at any location on the wearable device 140. Alternatively, the wearable device 140 can include an engagable portion 154 of the fastening system at selected locations on the device 140 at which variable measurements are desired. For example, an engagable portion 154 of the fastening system may be incorporated at the instep, ankle, and calf areas of the compressive pressure device 132 for measuring applied compressive pressure in those areas. In one particular variation, the entire surface, or selected areas, of the compressive pressure device fabric can be bulked by heat treatment to form a thin “blanket” of filaments. That “blanket” of filaments establishes a large number of loops which can be made to serve as the loop portion of a hook-and-loop type fastening system. Nylon yarns are particularly amenable to forming a blanket of loops when heated in this manner.

Depending on the type of sensor, the sensor may be attached using a hook-and-loop type fastening system to the inner surface (adjacent a wearer's skin) or to the outer surface of the wearable device 140. One advantage of attaching a sensor to the compressive pressure device 132 using a hook-and-loop type fastener is that the sensor-containing strip portion is pliable about the anatomical contours of a wearer's limb, such as about the ankle.

In one aspect of the present invention, changes in a variable are either sensed in the form of an electrical signal or are converted to an electrical signal. The electrical signal can be transmitted to the electronic display unit 118.

In some embodiments of the body monitoring system 110 and/or method, the sensor can comprise the electrical sensor circuit 120 adapted to measure one or more variables. The electrical sensor circuit 120 can be configured to amplify and filter a sensed variable signal to enhance and “clean up” the signal. The “cleaned up” signal can then be sampled by an analog-to-digital converter, and curve-fitting equations can be utilized to convert the digital signal into a measurement of the variable, for example, a measurement of force.

In some embodiments of the body monitoring system 110 and/or method, the electrical signal transmitting a variable measurement can be transmitted via the transmission circuit 116 adapted for such transmissions. In some embodiments, the sensing circuit 120 and/or the transmission circuit 116 can be printed or etched onto a portion of a piece of material 150 comprising a hook-and-loop type fastener engagable with the wearable fabric 140. Such printed circuits 120, 116 can then be secured to the wearable fabric 140 using the hook-and-loop type fastening system.

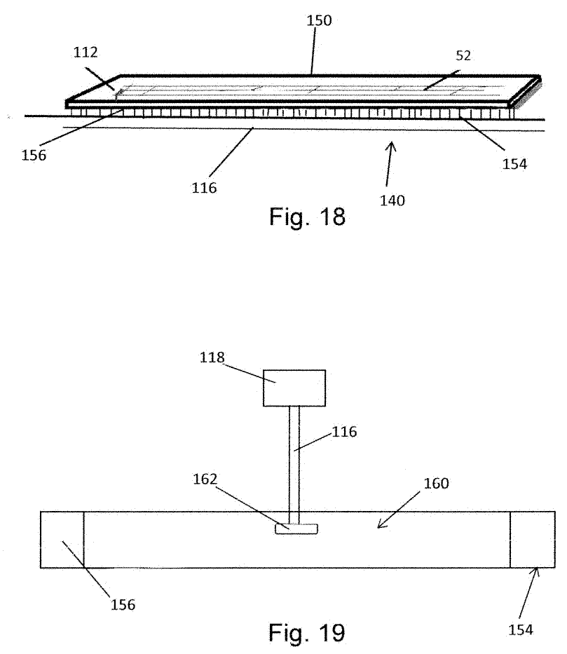

For example, as shown in the embodiment in FIG. 18, a piece of material 150 comprising the first portion 154 of a hook-and-loop type fastener can be printed with a sensor circuit 152 configured to sense a variable or parameter in/on a body. An electrically conductive yarn 112 can be sewn at a selected location in the sensor circuit 152 through the material 150 to expose the sewn conductive yarn 112 in the engagable first portion 154 of the hook-and-loop type fastener. The wearable fabric 140 can be constructed to comprise the second portion 156 of the hook-and-loop type fastener engagable with the first portion 154 of the fastener on the circuit material 150. The printed sensor circuit material 150 can be attached to the wearable fabric 140 at a location such that the exposed conductive yarn 112 on the circuit material 150 makes conductive contact with the transmission pathway circuit 116 in the fabric of the wearable device 140. In some embodiments, the body monitoring system 110 and/or method can comprise the body compression monitoring system 130 and/or method, the wearable device 140 can comprise the compressive pressure garment or device 132, and the printed sensor circuit 152 can be configured to sense applied compressive pressure.

The printed sensor circuit 152 can be placed against a body area to sense a variable. The sensor circuit material 150 and the printed sensor circuit 152 thereon can comprise a variety of shapes and/or dimensions. As a result, the printed sensor circuit 152 can be placed at various locations on a body while being connected to the transmission pathway circuit 116 in the wearable device 140. In this way, the printed sensor circuit 152 can be utilized to sense variables at particular locations in/on the body without having to vary the pathway of the transmission circuit 116. That is, one transmission pathway circuit 116 can be utilized to transmit signals from various, adjustable locations.

In some embodiments, the printed sensor circuit material 150 can be attached to a stretch fabric. Since the printed sensor circuit material 150 comprises a separate component from the knitted fabric to which it is attached, when the fabric is stretched, movement of the printed sensor circuit 152 is minimized and the ability of the printed circuit 152 to sense variables in a body is not affected. In some embodiments, the printed circuit 152 can be constructed so as to sense variable(s) and/or accept power from a power source.

In another aspect of the present invention, the body monitoring system 110 and/or method can comprise an adjustable pressurized cuff 160 that is wearable about a body area. As shown in FIG. 19, the pressurized cuff 160 can comprise an elongated piece of material, the ends of which can be overlapped onto each other and releasably connected. In the embodiment shown in FIG. 19, the cuff 160 can comprise a first portion 154 of a hook-and-loop type fastener on one end and a second, mating portion 156 of the hook-and-loop type fastener on the opposite end. The first and second hook-and-loop type fastener portions 154, 156, respectively, can be situated on the ends of the cuff 160 so that when the cuff 160 is wrapped about a circumferential surface, the ends of the cuff 160 can be releasably secured to each other about the surface so as to provide different lengths, and different amounts of tension, about the surface. The pressurized cuff 160 can further comprise one or more pressurized cuff sensors 162 integrated into the cuff 160 configured to sense pressure being applied by the cuff 160. The pressurized cuff sensor(s) 162 can be operably connected to the transmission circuit 116 that leads to the display unit 118.

In operation, the pressurized sensor cuff 160 can be placed about the person's limb 134, so as to overlie the compressive pressure garment 132 on the limb 134. The compressive pressure garment 132 can have a predetermined amount of compressive pressure when applied, for example, as calibrated on a tube having a particular circumference. Likewise, the pressurized sensor cuff 160 can be calibrated to provide a predetermined amount of compressive pressure when applied with a certain amount of tension. The pressurized sensor cuff 160 can be applied over the limb 134 and garment 132 so as to provide the same amount of compressive pressure as the amount rated for the garment 132. The amount of compressive pressure applied by the pressurized sensor cuff 160 can be adjusted by tightening or loosening the cuff 160 and securing the cuff 160 onto itself using the hook-and-loop type fastener system on the ends of the cuff 160. The amount of compressive pressure applied by a certain degree of tension on the cuff 160 can be monitored by reading the compressive pressure value displayed by the display unit 118. Thus, for a compressive pressure garment rated for 30 mm Hg pressure, for example, the pressurized sensor cuff 160 can be adjusted about the limb 134 and underlying garment 132 so that the display unit 118 displays an initial compressive pressure value of 30 mm Hg. As the amount of applied compressive pressure on the limb 134 changes, the amount of pressure within the pressurized sensor cuff 160 changes proportionately. For example, as the girth of the limb 134 increases due to increasing edema, the amount of compressive pressure being applied by the pressure garment 132 and by the pressurized sensor cuff 160 increase. Accordingly, the display unit 118 will display an increasing compressive pressure value, thereby alerting the patient and/or caregiver that the actual applied compressive pressure may be too high for therapeutic purposes.

The sensor can be placed between a patient's body and the wearable device 140, such a compressive pressure sleeve, such as the sleeve 142 shown in FIG. 20. In such a configuration, the sensor can measure the cumulative, or total, compressive pressure applied by both the sleeve 142 and any overlying garment, such as the compression wrap 144. Alternatively, the sensor can be placed between the sleeve 142 and the overlying compression wrap 144 such that the sensor measures only the compressive pressure applied by the overlying wrap 144. In such an embodiment, the sleeve 142 having a predetermined applied compressive pressure, for example, about 5 mm Hg compressive pressure, can be placed on the patient's limb 134. The sensor can be attached to the outer surface of the sleeve 142 prior to the sleeve 142 being placed on the patient's limb 134, or the sensor can be placed on the outer surface of the sleeve 142 after the sleeve 142 is placed on the patient's limb 134. The wrap 144 can then be applied over the sensor and sleeve 142 such that the sensor is positioned between the inner sleeve 142 and the outer wrap 144. Once the outer wrap 144 is applied, the sensor can measure the compressive pressure applied by the outer wrap 144. By knowing the actual pressure applied by the wrap 144 on the patient's limb 134, the wrap 144 can be loosened or tightened to achieve a desired cumulative, or total, compressive pressure applied by both the inner sleeve 142 and the outer wrap 144. For example, if the total compressive pressure desired for treatment of a venous leg ulcer underneath the sleeve 142 and wrap 144 combination is 40 mm Hg pressure, the wrap 144 can be adjusted to provide 35 mm Hg pressure as measured by the sensor, which combined with the 5 mm Hg pressure provided by the sleeve 142 achieves the desired cumulative compressive pressure. In this way, the actual initial compressive pressure applied by the wrap 144, or sleeve 142 and wrap 144, for a particular treatment can be achieved with some certainty.

In another embodiment in which the sensor is place between the sleeve 142 and the wrap 144, the sensor can be configured to sense the actual compressive pressure at the interface between the patient's body, the sleeve 142, and the wrap 144. In either configuration—those in which the sensor is placed between the body and the sleeve 142 or those in which the sensor is placed between the sleeve 142 and the wrap 144—the sensor can sense changing pressure in the body area being monitored. In this way, the patient and/or caregiver can readily determine the actual amount of applied compressive pressure at any time and make adjustments as needed.

Embodiments of the body monitoring system 110 allow sensors to be positioned at various and multiple locations in the wearable device 140. For example, sensors can be positioned at the instep, ankle, calf, and other anatomical locations. As a result, real-time measurements of the variable(s) can be monitored simultaneously across the entire wearable device 140. Such flexibility in measurement allows the benefit of monitoring, for example, actual applied compressive pressures along a graduated compression device.

In addition, the compressive pressure sensor can be adapted to take measurements of applied compressive pressure at multiple points within a particular sensor field. For example, the knitted cuff sensor 124 or the knitted-in sensor circuit 120 having a horizontal configuration can take measurements of applied compressive pressure simultaneously at multiple points about a circumference of the limb 134 on which the device 132 is being worn. Averaged measurements of applied compressive pressure provide the advantage of increased accuracy over individual point measurements. Thus, such multiple point measurements of compressive pressure can be averaged to provide a more accurate representation of actual compressive pressure being applied across a defined area.

In some embodiments, variables measured by a sensor can be transmitted to a data display, processing, and/or recording device 118. Various mechanisms can be utilized to display, process, transmit, and/or record measurements of the sensed variable(s). In some embodiments, variable data can be transmitted from a point of measurement to a miniature microprocessor and display unit 118 attached to the wearable device 140. The miniature display unit 118 is preferably an electronic display unit 118, for example, a miniature LCD or LED display screen.

The electronic display unit 118 can be attached to the wearable device 140 in various ways and locations. In one embodiment, the display unit 118 can be attached to the wearable device 140 using a clamping mechanism. In another embodiment, the wearable device 140 can be knit at a desired location on the device the cuff 122 or pocket 126 for housing the display unit 118. For example, the pocket 126 can be knit at the top, or proximal end, of a compressive pressure stocking, for example. The display unit 118 can be placed inside the pocket 126 such that the display unit 118 does not touch the patient's body.

The electronic display unit 118 can display the amount of compressive pressure actually being applied in a particular sensing area at any given time. In this way, persons managing compressive pressure therapy can adjust the compressive pressure device 132 while attending the patient without having to review the data at another location. Alternatively, or in addition, such data can be transmitted wirelessly to a computer at another location. Recording transmitted compressive pressure data can beneficially provide a clinical record of compressive pressure therapy for a patient over time. Such data display, transmission, and/or recording mechanisms 118 can be utilized with any embodiment of a body monitoring system 110 according to the present invention.

Some embodiments of the body compression monitoring system 130 can include compression level alarms. For example, if actual compressive pressure falls below a set minimum threshold, the system can trigger a low pressure alarm. That is, if actual applied compressive pressure drops below a certain level due to decrease in edema underneath the compressive pressure device, fabric fatigue, or other reason, the system can send a signal (visual and/or auditory) to the local display unit 118 and/or to a remote location that the pressure is too low. The system 110 can also provide a high pressure alarm that similarly alarms when pressure becomes too high, such as when the device 132 slips out of position or edema increases. Embodiments of the body monitoring system 110 and/or method provide a mechanism for accurately determining an actual amount of compressive pressure applied by a compressive pressure device to a patient. Such a body compression monitoring system 130 and/or method can provide accurate measurements of compressive pressure applied over the entire area or in selected areas underneath the compressive pressure device 132. Such a body compression monitoring system 130 and/or method can provide accurate measurements of applied compressive pressure the entire time the device is being worn.

In some embodiments, measurement and/or recording of the variable(s) can be continuous or at selected intervals. Such dynamic clinical information facilitates the administration of therapeutic amounts of compressive pressure, for example, so as to achieve desired outcomes. Accordingly, as a result of such accurate and ongoing information, systems and methods according to the present invention can facilitate optimized care in the treatment and prevention of vascular and other conditions.

In addition, documentation of actual applied compressive pressure can enhance risk management related to clinical practice, and can a record of treatment for reimbursement purposes.

Embodiments of the body compression monitoring system 130 and/or method can be easily utilized by clinicians, as well as by patients or other non-clinicians.

Embodiments of the body compression monitoring system 130 and/or method can be utilized in combination with other compression therapy devices. For example, the body compression monitoring system 130 can be utilized in combination with stockings, hosiery, sleeves, wraps, bandages, and/or other means for providing compression therapy. Some embodiments can be positioned adjacent a wearer's skin with another compression therapy garment overlying the body compression monitoring system 130. In other embodiments, the body compression monitoring system 130 can be applied over another compression therapy garment. In either case, the body compression monitoring system 130 can be utilized to accurately monitor compressive pressure actually applied by the combination of compression therapy means.

Embodiments of the body monitoring system 110 and/or method provide a mechanism for accurately measuring body variables regardless of variables related to yarn, fabric construction, stretch characteristics, number of fabric layers, yarn/fabric fatigue, body shape and circumference, and other variables related to a therapeutic wearable device (such as wearable device 40) and its application.

Some embodiments of such a body compression monitoring system 130 and/or method may be useful for allowing a user to easily and accurately determine compressive pressure at different locations on a person's body. In such a body compression monitoring system 130 and/or method, accurate measurements of applied compressive pressure at various anatomical locations, for example, along a leg, can provide assurance that compressive pressures are appropriately graduated.

Various embodiments of the body compression monitoring system 130 and/or method can be utilized on different anatomical areas. For example, some embodiments of the body compression monitoring system 130 and/or method can be utilized to monitor compressive pressure applied to a leg in treatment of venous insufficiency or a venous ulcer. Other embodiments can be utilized to monitor compressive pressure applied to an arm in treatment of lymphedema. Yet other embodiments can be utilized to monitor compressive pressure applied to a chest following breast surgery or to an abdomen after a liposuction procedure. The range within which actual applied compressive pressure may be accurately monitored can vary, depending on the amount of compression to be applied by a device. For example, the range of compressive pressure to be applied in treatment of lymphedema in an arm may be higher than the range of compressive pressure to be applied in treatment of venous insufficiency in a leg. Accordingly, the range within which actual applied compressive pressure may be accurately monitored in the lymphedema application would be greater than that for a venous insufficiency application.

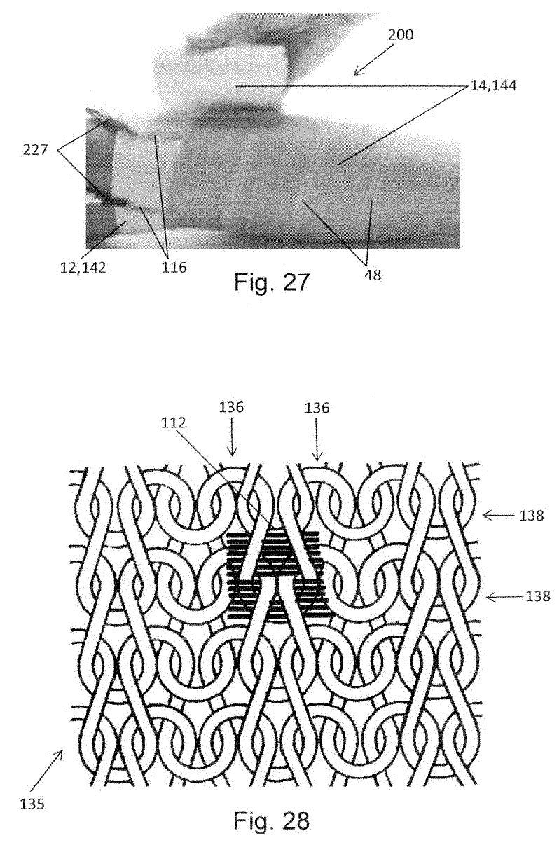

The subject matter described herein includes embodiments of a compression and sensing system and/or method. Some embodiments of such a compression and sensing system 200 and/or method comprise a wearable device, such as the compressive pressure device 132 shown in FIG. 11; a sensor 220 connected to the wearable device 132 and configured to sense compressive pressure in an area of a body to which the device 132 is applied; and the transmission circuit 116 configured to conduct, or transmit, an electrical signal representing a compressive pressure value in an area of a body to another location. In some embodiments, the wearable device 132 can comprise an elastic fabric. Such a compression and sensing system 200 is illustrated in FIGS. 21-27

Alternatively, the subject matter described herein includes embodiments of a sensing system and/or method other than a system or method that senses compression. In such embodiments, the sensor can be configured to sense one or more other variables in an area of a body to which the device is applied. Likewise, the transmission circuit 116 can be configured to transmit an electrical signal representing a value(s) of the variable(s) sensed in an area of a body to another location.

In preferred embodiments, the transmission circuit 116 comprises an electrically conductive yarn 112 knitted into the device 132. The electrically conductive yarn 112 can be knit in any direction, either vertically, horizontally, or at an angle. Preferably, the electrically conductive yarn transmission circuit 116 is knit in a vertical direction along the length of the wearable compression device 132. The direction and specific path of the transmission circuit 116 can be determined by the selection of stitch pattern and conductive yarn.

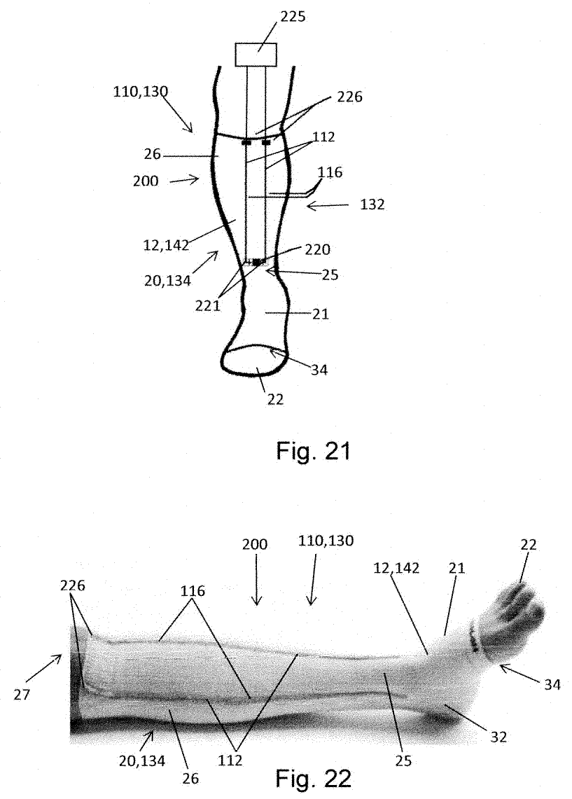

In some embodiments, the wearable device 132 comprises a compressive pressure device. In a preferred embodiment, the compressive pressure device 132 comprises an inner compressive pressure sleeve 12, 142 and an overlying outer compressive pressure wrap 14, 144, as shown in FIGS. 22, 26, and 27. The fabric sleeve 12, 142 acts as a first layer of the compressive pressure device 132, and can be constructed to fit a limb (arm or leg (20, 134)) with minimum compression, for example, about 5-10 mm Hg of compressive pressure. Once the inner sleeve 12, 142 is placed on a patient, the outer wrap 14, 144 can be placed over the inner sleeve 12, 142. Various commercially available compressive pressure wraps can be utilized as the outer wrap 14, 144 in embodiments of such a compressive pressure device 132 according to the subject matter described herein. One or more outer wraps 14, 144 can be applied.

Compressive pressure can be measured with the sensor 220 when the sleeve 12, 142 is placed on a patient's body, and then again when the wrap 14, 144 is placed over the sleeve 12, 142. Such measurements provide certainty of the actual applied compressive pressure(s) when the sleeve 12, 142 or sleeve 12, 142 and wrap 14, 144 are applied. In such an embodiment of an inner sleeve-outer wrap system, the sensor 220 can be located either (a) between the body and the sleeve 12, 142, (b) within the sleeve 12, 142, (c) between the sleeve 12, 142 and the wrap 14, 144, or (d) within the wrap 14, 144. In either of these locations, the sensor 220 is configured to sense an actual cumulative amount of compressive pressure applied by the sleeve 12, 142 and the wrap 14, 144. By knowing the actual pressure applied by the wrap 14, 144 on the patient's limb (20, 134), the wrap 14, 144 can be loosened or tightened to achieve a desired cumulative, or total, compressive pressure applied by both the inner sleeve 12, 142 and the outer wrap 14, 144.

In some embodiments, the location to which the electrical signal representing a compressive pressure value is transmitted comprises an external device separate from the wearable device. For example, the external device can be connected to the transmission circuit and can comprise a data processor and/or an electronic display unit 225 (as shown in FIG. 21) configured to display the transmitted compressive pressure value.

In one aspect of the subject matter described herein, embodiments of the electrically conductive yarn 112 knit into the compression device 132 as the transmission circuit 116 comprise yarns having a high number of filaments/fibers. In preferred embodiments, the electrically conductive transmission circuit yarn 112 comprises a 70 denier yarn having from about 24 to about 68 filaments/fibers.

A higher number of filaments/fibers in this range provides for entanglement and greater contact between the filaments/fibers within the conductive yarn 112 and between conductive yarns 112. This greater yarn contact along the conductive yarn 112 in the transmission circuit results in decreased resistance along the circuit 116. Resistance is an electrical quantity that measures the degree to which a device or material reduces flow of electric current through it. Resistance is measured in units of ohms (Q). The lower the resistance, the greater the conductivity. That is, as the yarn filaments/fibers interact, resistance is decreased and conductivity is enhanced.

The resistance (or resistivity, of which conductivity is the reciprocal) along the transmission circuit 116 of the knitted structure of the compression device 132 is preferably about 50 ohms or less per 10 cm. Embodiments of the electrically conductive yarn 112 comprising a 70 denier yarn having from about 24 to about 68 filaments/fibers provides a resistance (or conductivity) along the transmission circuit 116 between about 20 ohms to about 2 ohms per 10 cm. Accordingly, a 70 denier conductive yarn 112 having from about 24 to about 68 filaments/fibers provides optimal conductivity for transmitting electrical signals representing a compressive pressure value along the transmission circuit 116 in the compression device 132.

In one preferred embodiment, the compression and sensing system 200 and/or method can comprise the compression device 132 having a terry knit construction on the back (inner) side of the sleeve 12, 142. In such an embodiment, the conductive yarn 112 can comprise four 70 denier nylon yarns, each wrapped with a 24 filament silver yarn and twisted together. The conductive yarns 112 can be air entangled to provide conductivity for each yarn in the range of about 10 ohms per 20 cm. In such an embodiment, the conductive yarn 112 can be spliced-knit with one or more needles to provide the conductive yarn 112 laid in one side of the fabric structure. The conductive yarn 112 can be laid in on each revolution of the circular knitting machine. For example, using a terry knit pattern, the conductive yarn is splice-knit under the sinker, while the terry layer yarn is knitted over the sinker. This produces a terry loop pattern on one (inner) side of the fabric with the conductive yarn cut and laid in, or “floated,” vertically to form the transmission circuit 116. In such an embodiment, a desired conductivity can be maintained in the transmission circuit 116, while reducing the likelihood (in higher filament yarns) of filaments protruding through the terry layer and making contact with the body.

As shown in FIG. 28, one or more high filament conductive yarns 112 can be laid in within a jersey 135 or cushion fabric knit structure. The laid-in yarn 112 can be one or more stitches wide. The conductive yarn 112 can be knitted in every revolution of the circular knitting machine and cut to a desired width.

In such embodiments in which the conductive yarn 112 is laid-in to the fabric structure, the effect on conductivity by horizontal stretch in the compression device 132 is negligible. In addition, when placed on a patient, for example, in the compression device 132, the higher number of filaments/fibers in such an embodiment pack together, thereby enhancing conductivity in the transmission circuit 116. Thus, the change in resistance read by the data processor 225 is that measured by the sensor 220, which is not affected by any minimal change in resistance due to stretching in the transmission circuit conductive yarn 112.

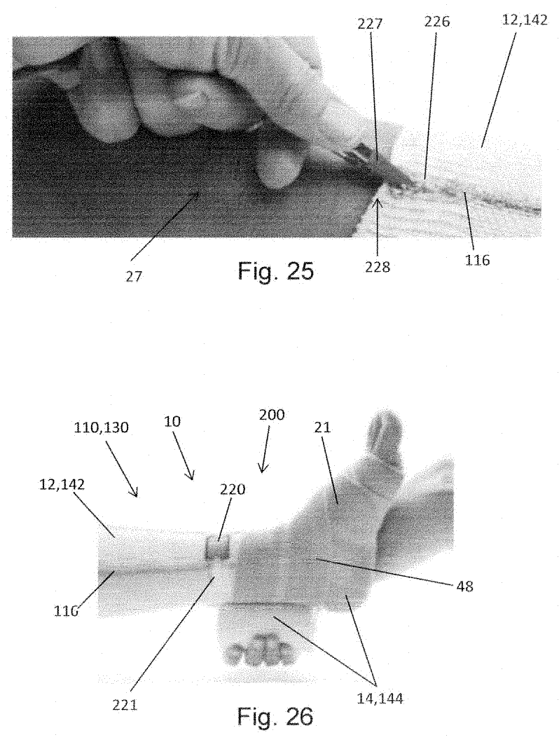

One disadvantage of using a conductive yarn 112 having a larger denier is that such a yarn can create a ridge of undesired point pressure on a patient when the compressive pressure device 132 is worn. The smaller denier conductive yarns 112 in such preferred embodiments, especially when used in conjunction with a terry knit interior of the compressive device 132, do not cause a line of point pressure on a patient. However, in certain embodiments, the conductive yarn 112 can be wider at the top 228 of the sleeve 12, 142 than in the remainder of the transmission circuit 116. A wider terminal portion of the conductive yarn 112 provides a more secure connection point 226 for the display unit connector 227, as shown in FIG. 25.

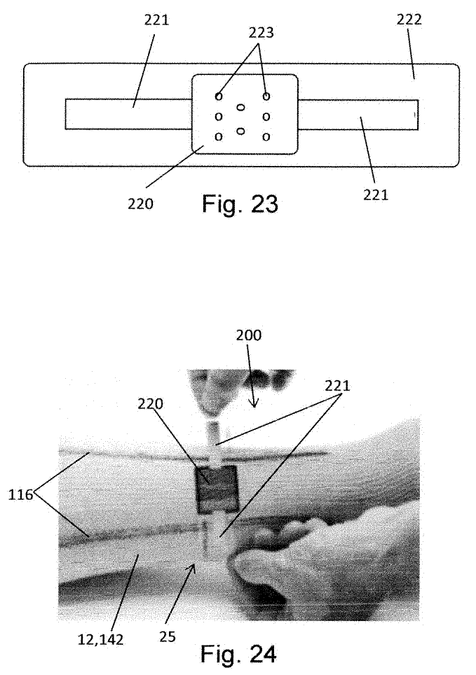

In another aspect of the subject matter described herein, embodiments of the compression and sensing system 200 and/or method can comprise various sensor configurations. In one embodiment, the compression and sensing system 200 and/or method comprises the pressure sensitive sensor 220, as shown in FIGS. 23, 24, and 26. The pressure sensitive sensor 220 can be constructed having a sensing area of approximately 2.5 cm×2.5 cm. An electrical connection 221 comprising a metallic strip extends in the same plane from each side of the sensing area. One of the metallic strip electrical connections 221 is a positive terminal, and the opposite metallic strip electrical connection 221 is a negative terminal. Each electrical connection 221 is designed to connect to a separate conductive yarn 112 in the transmission circuit 116. In some embodiments, one side of each electrical connection 221 can be insulated and the other side configured to make contact with one of the conductive yarns 112 in the transmission circuit 116. For example, the pressure sensitive sensor 220 can be placed on the outside of the compression device 132, such as the compression sleeve 12, 142, to connect to the transmission circuit 116.

In a preferred embodiment, the pressure sensitive sensor 220 further comprises an adhesive backing 222 with a protective cover over the adhesive. The protective cover can be removed to adhere the adhesive backing 222, and the sensor 220, onto the outer surface of the compression device 132, such as the compression sleeve 12, 142. The adhesive backing 222 can be configured so as to adhere the sensor 220 to the compression device 132 without allowing adhesive to contact the transmission circuit conductive yarns 112.

In some embodiments, the sensor 220 comprises a capacitive-type pressure sensor, or capacitive touch sensor, as shown in FIGS. 23, 24, and 26. The capacitive pressure sensor 220 is configured to measure the actual applied interface pressure delivered by the compression device 132, such as the compression sleeve 12, 142 and/or the compression wrap 14,144.

In an alternative embodiment, one or more of the capacitive pressure sensors 220 can be incorporated into a plastic strip. The plastic strip can be applied to the compressive pressure device 132. Each sensor in the plastic strip is attached to a separate one of the transmission circuits 116 extending to one end of the plastic strip. A measure of interface compressive pressure sensed by each sensor 220 in the plastic strip is transmitted via one of the transmission circuits 116 to the end of the plastic strip, where a data processor and/or display unit (such as processor 225) can be attached. Each sensor 220 can be read separately with the same data processor and/or display unit 225.

When the sensor 220 has a flat surface, interface pressure applied by an air bladder pressure cuff can be measured accurately. However, it was discovered that an overlying compression garment, such as the compression wrap 14, 144, applies pressure differently than that applied by an air bladder pressure cuff. An air bladder exerts an evenly distributed force on the sensor 220, whereas when the overlying compression wrap 14, 144 is applied to a body, yarns/fibers in the overlying garment 14, 144 pull unevenly across the sensor 220. Thus, in some embodiments, the sensor 220 can include an interface extender that extends slightly above the surface, or plane, of the sensing area in the sensor 220 in order to re-distribute force from the overlying garment 14, 144 more evenly. In this way, compressive pressure provided by the overlying garment 14, 144 can be accurately measured. In one embodiment, the sensor interface extender can comprise, for example, a layer of material placed between the sensor surface and the overlying fabric. In other embodiments, the sensor interface extender can comprise an alteration in the surface of the sensor. For example, the shape of the sensor surface can be altered to extend slightly upward, such as in a convex manner to interface with the overlying compression fabric.

In a preferred embodiment of the compression and sensing system 200 and method, the sensor 220 includes an interface extender comprising a plurality of spaced apart projections 223, such as rounded bumps, or bubbles, extending slightly outward from the sensing surface of the sensor 220. The projections 223 can have a size and pattern that engages the curvature of a patient's leg 20, 134 when attached to the compression sleeve 12, 142. In this way, the sensor 220 can maintain a constant and even contact with transmission circuit 116.

In some embodiments of the compression and sensing system 200 and method, a plurality of the pressure sensitive sensor 220 can be placed on the compression device 132. For example, one of the pressure sensitive sensors 220 can be placed on the foot 21, one on the ankle 25, and one on the calf area 26 of a patient. Each sensor can be connected to and read by the same data processor and/or display unit 225.

In a particular embodiment, a first sensor 220 can be connected to the transmission circuit 116 at the ankle 25 and a second sensor 220 can be connected onto the same transmission circuit 116 yarns 112 at a second location, for example, at the calf 26 of the leg 20, 134. The data processor/display unit 225 can be configured to read and display the compressive pressures at both locations, the signals for each compressive pressure transmitted by a single transmission circuit 116. In yet another particular embodiment, the compressive pressure device 132 can include a first transmission circuit 116 and a second transmission circuit 116. In this embodiment, a first sensor 220 can be connected to the first transmission circuit 116 at, for example, the ankle 25, and a second sensor 220 can be connected to the second transmission circuit 116 at, for example, the calf 26. The data processor/display unit 225 can be configured to read and display the compressive pressures at both locations, the signal for each location compressive pressure transmitted by separate transmission circuits 116.

In another aspect of the subject matter described herein, embodiments of the compression and sensing system 200 and/or method can comprise the data processor/display unit 225, such as a computer, connectable to a connection point 226 on the transmission circuit 116. In some embodiments, the data processor/display unit 225 can be disconnected from the transmission circuit 116 and reconnected for further measurements. Alternatively, the data processor/display unit 225 can be permanently attached to the transmission circuit 116 such that continuous compressive pressure readings can be provided.

In some embodiments, the sensor 220 can be configured to sense an amount of electrical resistance indicative of a particular level of compressive pressure. Resistance is measured in ohms, which is transmitted to the data processor 225. The data processor 225 can convert the level of ohms to an amount of compressive pressure, expressed in mmHg. The data processor can be programmed to disregard electrical activity in the range produced by a body's natural conductivity.

In embodiments of the compression and sensing system 200 and/or method in which the sensor 220 comprises a capacitive-type pressure sensor, a capacitance value measured by the sensor 220 can be transmitted to the data processor 225, where the capacitance value can be correlated with a particular level of compressive pressure.

The accuracy and reliability of compressive pressure measurements by the compression and sensing system 200 was tested. The compression and sensing system 200 is also known as the SMART SLEEVE® to be commercially available from Carolon Company, 601 Forum Parkway, Rural Hall, N.C. 27045. In one experiment, measurements by the compression and sensing system 200 were compared with measurements by a PICOPRESS® compression measurement system, which has been shown to provide reliable measurements of compressive pressure. A PICOPRESS® compression measurement system utilizes a pneumatic pressure transducer to measure pressure exerted by an overlying compressive device, such as a sleeve, wrap, or bandage, onto a sensor applied to a patient's body. The PICOPRESS® system is commercially available from mediGroup Australia Pty. Ltd., lvl 1, 530 Little Collins Street, Melbourne VIC 3000 Australia.

In this experiment, a PICOPRESS® sensor was placed on a person's lower leg 20, 134 and attached to its reader. The inner sleeve 12, 142 was applied onto the lower leg 20, 134, and the sensor 220 was attached to the inner sleeve 12, 142 and transmission circuit 116 at the same level on the lower leg as the PICOPRESS® sensor. The data processor/display unit 225 of the compression and sensing system 200 was connected to the transmission circuit 116. Then, the outer wrap 14, 144 was applied over the inner sleeve 12, 142 and both sensors so that the compression and sensing system 200 data processor/display unit 225 indicated compressive pressure of 25 mm Hg. At this measurement by the compression and sensing system 200, the PICOPRESS® reader indicated compressive pressure of 27 mm Hg. Then, the outer wrap 14, 144 was adjusted so that the compression and sensing system 200 data processor/display unit 225 indicated compressive pressure of 5 mm Hg higher than the previous reading (i.e., 30 mm Hg), and a reading of compressive pressure by the PICOPRESS® system was taken. This step was repeated five more times such that compressive pressure was increased in 5 mm Hg increments (to a total of 55 mm Hg pressure) according to the compression and sensing system 200 data processor/display unit 225. A reading by the PICOPRESS® system was taken at each incremental level of compressive pressure.

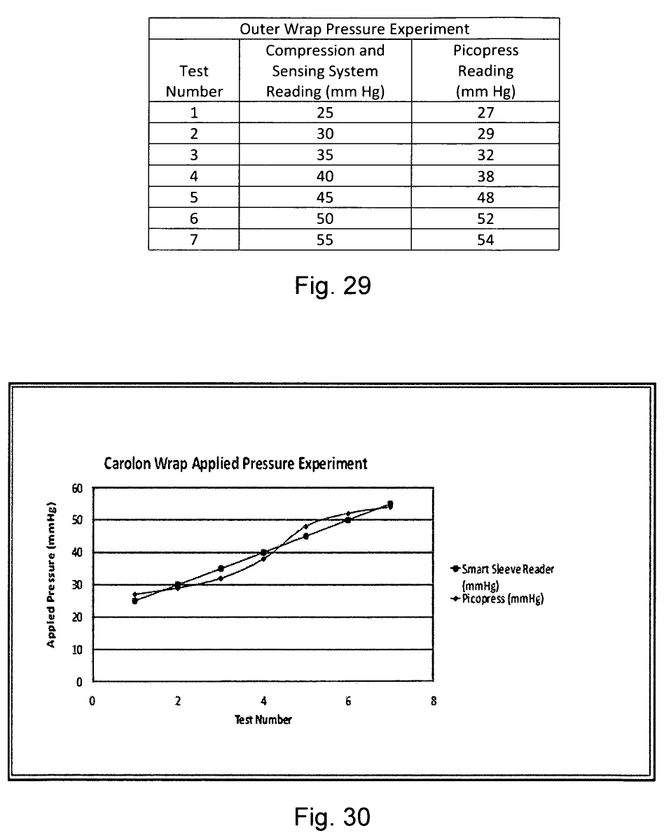

FIG. 29 shows the results of this experiment in table form, and FIG. 30 graphically indicates the correlation between measurements by the compression and sensing system 200 and the PICOPRESS® system. These results show that measurements of compressive pressure by the compression and sensing system 200 correlate closely with measurements by the PICOPRESS® system at each level tested. Accordingly, measurements of compressive pressure by the compression and sensing system 200 are shown to be accurate and reliable.

Some embodiments of the compression and sensing system 200 and/or method can comprise a means for housing the transmission circuit connection points 226 and/or the data processor/display unit 225. Such housing means can provide protection against contamination of the transmission circuit connection points 226 and data processor/display unit 225 from wound drainage or other sources. When the data processor/display unit 225 is utilized with more than one patient, such protection of this hardware helps minimize the risk of cross-contamination with other patients. In some embodiments, the housing means can comprise a cuff integrally knit in the wearable compression device 132, for example, at the top 228 of a lower limb compression sleeve 12, 142. In other embodiments, the compression device 132 can include a portion of fabric that can be turned back onto the device 132 so as to create a covered space. Such housing means can keep the connection points 226 and data processor/display unit 225 from touching a patient's skin and reduce the risk of cross contamination, as well as protect the patient's skin from contact and possible irritation by those components.

In some embodiments, the transmission circuit connection points 226 can be located at the top of the compression device 132, for example, at the top of a lower limb compression sleeve 12, 142, as shown in FIGS. 22, 25, and 27. In this way, the connection points 226 can be easily cleaned, further enhancing protection against contamination.

In certain limited situations, it may be possible that the skin of a patient's body will conduct current so as to effectively “short circuit” the transmission circuit 116. As a result, non-insulated transmission circuit conductive yarn 112 in direct contact with the body may affect reliable transmission of an electrical signal representing a compressive pressure value from a sensor. Thus, in another aspect of the subject matter described herein, embodiments of the electrically conductive yarn 112 knit into the compression device 132 as the transmission circuit 116 can be insulated from the body of a wearer.

Insulation of conductive yarns 112 from the body can be achieved in several effective ways. For example, the knitted compression device 132 can be constructed so that transmission circuit conductive yarns 112 are located on the outside of the fabric structure and non-conductive, insulating yarns are located on the inside of the fabric structure. In this way, when the device 132 is placed on a wearer, the inner non-conductive yarns provide insulation between the outer conductive yarns 112 and the wearer's body. The insulating portion of the fabric structure can be constructed about the entire inside surface of the compression device 132. Alternatively, the insulating portion can be constructed only underneath the portion of the compression device 132 comprising the transmission circuit 116. The inner insulating portion of the fabric structure can be knit by floating non-conductive yarns or by knitting a pattern of non-conductive yarns behind, or underneath, the transmission circuit 116. Preferred non-conductive yarns useful in insulating transmission circuit conductive yarns include nylon, rayon, polyester, and cotton. In other embodiments, transmission circuit conductive yarns 112 can be insulated by wrapping the conductive yarns with one or more non-conductive yarns or fibers, such as nylon.

In other embodiments, a layer of non-conductive material can be placed between the transmission circuit conductive yarns 112 in the compression device 132 and the body of a wearer. For example, the transmission circuit conductive yarns 112 in the compression device 132 can be insulated from the body by placing a non-conductive stocking or sleeve on a wearer underneath the compression device 132. Alternatively, the compression device 132 can be constructed (such as in the form of a sleeve) so that a portion of the device fabric structure comprising non-conductive yarns can be folded back onto/underneath a portion having transmission circuit conductive yarns 112. In both approaches, the layer of non-conductive material insulates the transmission circuit conductive yarns 112 from the body of a wearer.

In some embodiments, the thickness of insulating yarns is equivalent to that of at least about a 30 denier yarn so as to provide sufficient insulation to avoid short-circuiting of the conductive yarns 112. Such a degree of insulation can be provided with one or more layers of insulating yarn/fabric.

In certain embodiments, selected areas in the compression device fabric structure can include conductive yarns 112 to provide transmission circuit pathways 116 from one or more sensor sites in the device 132 to a location for connection with the data processor/display unit 225. Sensor sites can be connected together in serial fashion via the transmission circuit 116 for ultimate connection to the data processor/display unit 225, or each sensor site can be separately connected in parallel by individual transmission circuits 116 to a location for connection with the data processor/display unit 225. In each sensor-transmission circuit design, conductive yarns in each transmission circuit 116 can be insulated from a wearer's body using one or more of the insulation techniques described herein.

In another embodiment of the compression and sensing system 200 and/or method, the transmission circuit 116 can be comprised in a strip of a hook-and-loop type fastener. For example, a first portion, or strip, of a hook-and-loop type fastener material can comprise conductive material configured to define the transmission circuit 116. Alternatively, a first portion, or strip, of a hook-and-loop type fastener material can comprise conductive yarn 112 sewn into the material so as to define the transmission circuit 116. A compression device fabric can be constructed to comprise the second portion of the hook-and-loop type fastener engagable with the first portion of the fastener material. In this manner, the first conductive portion of a hook-and-loop type fastener material can be attached to the compression device 132 in a desired location to provide the transmission circuit 116 between a sensor and a hardware connection point. By attaching the first conductive portion of a hook-and-loop type fastener material to the outside of the compression device 132, the transmission circuit 116 can be insulated from a wearer's body by the underlying compression device structure.

In another aspect of the subject matter described herein, in embodiments of the knitted compression device 132, conductive yarns 112 and insulating yarns can be knit in the same circular knitting process.

Conductive yarns 112 for the transmission circuit 116 can be knit into the wearable compression device 132 while the device is being knit. In an exemplary embodiment, the conductive yarns 112 are knit on one or more needles along a wale or a selected number of adjacent wales along the longitudinal axis of the device 132. Such a “vertical” transmission circuit pathway 116 can be knit using various knit patterns, such as a rib pattern.

During the process of knitting, the conductive yarns 112 knit in the vertical transmission circuit 116 are preferably “packed” together vertically. That is, the conductive yarns 112 are knit tightly so that the stitch loops in adjacent courses along a particular wale are compacted together so as to have sufficient yarn/fiber contact to provide a continuous circuit and desired conductivity. Such a continuous circuit allows transmission of an electrical signal representing a compressive pressure measurement from a sensor in the compression device 132, such as at the ankle 25, vertically to another location, such as to a connection with the data processor/display unit 225 at the top of the device.

In some embodiments of the knitted compression device 132, the conductive yarns 112 and insulating yarns can be knit in the same knitting process on a circular knitting machine. In this way, conductive yarns in the transmission circuit 116 can be readily and economically provided with insulation from a wearer's body when the device 132 is applied. For example, a non-insulated conductive yarn can be knitted into the compression device 132 while insulating yarns are also being knitted by manipulating yarn feeds to produce a conductive yarn pattern on one (outer) side of the fabric and a non-conductive, insulating yarn pattern on the other (inner) side of the fabric.

In some embodiments, the conductive yarn 112 can be spliced-knit with one or more needles to provide the conductive yarn 112 laid in one side of the fabric structure. The conductive yarn 112 can be laid in on each revolution of the circular knitting machine. For example, using a terry knit pattern, the conductive yarn 112 is splice-knit under the sinker, while the insulating yarn is knitted over the sinker. This produces a terry loop pattern of insulating yarn on one (inner) side of the fabric, which can insulate the splice-knit conductive yarn 112 on the other (outer) side of the fabric when applied to a body. The tension of the insulating yarns can be varied to produce more or less fabric in the insulating yarn side of the fabric.

In embodiments in which the conductive yarns 112 are individually wrapped with an insulating yarn, the insulated conductive fiber can be laid in or knitted in a fabric structure either in the warp direction or weft direction.

Embodiments of the compression and sensing method can comprise methods of making and using compression and sensing systems, according to the subject matter described herein. As a particular example, such a method can include: (1) applying the compression device sleeve 12, 142 having the conductive transmission circuit 116 to a lower leg 20, 134; (2) aligning the transmission circuit 116 on the front of the lower leg 20, 134 (along the anterior tibial crest); (3) positioning the distal sensor connection area of the transmission circuit 116 at the smallest ankle circumference; (4) attaching the compressive pressure sensor 220 to the outside of the compression device sleeve 12, 142 so that the sensor terminals contact the transmission circuit conductive yarns 112; (5) connecting the data processor/display unit 225 to the proximal connection points 226 on the transmission circuit 116; (6) reading on the display unit 225 a first measurement of compressive pressure provided by the compression device sleeve 12, 142; (7) applying the a compressive wrap 14, 144 over the compression device sleeve 12, 142; and (8) reading on the display unit 225 a second measurement of the cumulative compressive pressure provided by the compression device sleeve 12, 142 and the compressive wrap 14, 142.

Embodiments of a compression and sensing system and/or method as described herein can provide advantages over conventional compression/sensing systems. For example, such a system provides a means for easily and accurately determining an actual amount of interface compression applied at an anatomical area by a compressive pressure device. As a result, the actual compressive pressure applied by a compression device can be utilized to verify compressive pressure within a desired therapeutic range.

Another advantage is that such a system provides a means for easily and accurately determining an actual amount of applied compressive pressure continuously while the device/garment is being worn.

Another advantage is that such a system provides a means for easily and accurately determining an actual amount of applied compressive pressure that is reliable across repeated measurements.

Another advantage is that such a system provides a means for easily and accurately determining an actual amount of applied compressive pressure when adding multiple layers of compressive material. Adding multiple layers of compressive material, for example, the outer wrap 14, 144, can have a multiplier effect on cumulative compressive pressure greater than the sum of pressures provided by each individual wrap in a single layer. As a result, multiple layers of compressive material can generate an unexpectedly high cumulative compressive pressure that can create undesired effects in a patient. Accordingly, it is important to measure cumulative compressive pressure as each subsequent layer of compressive material is added to a patient's body.

Another advantage is that such a system provides a means for easily and accurately determining an actual amount of applied compressive pressure that is economically constructed, including relatively inexpensive sensors, compression devices having a transmission circuit, and data processors and display units.

Another advantage is that such a system provides a means for easily and accurately determining an actual amount of applied compressive pressure that decreases risk for cross contamination. In some embodiments, each of the sensor, compression device, and hardware are usable by a single patient and disposable.

Another advantage is that embodiments of the compression and sensing system and method allow the provider the unique ability to adjust, measure, and document actual applied compressive pressure, and to downgrade the pressure as needed to maintain perfusion in a patient's limb.

Such advantages further allow clinicians to follow standards of care in compression therapy. For example, according to published guidelines, points during compressive pressure therapy that compression levels should be measured include: (1) during initial application to obtain a selected therapeutic pressure; (2) during each subsequent visit to the provider; (3) prior to removal of a bandage dressing for wound inspection; (4) during application of a new dressing; and (5) prior to removal of a wound dressing at the end of treatment.

In another aspect, the compression tension adjusting, stabilization, and sensing system and method comprises a tension adjusting and stabilization system and method, as shown in FIGS. 1-5 and 31-38.

In such a tension adjusting and stabilization system and method 300, the elastic compression bandage, or wrap, 14 comprises longitudinal, or “long-stretch,” elastomeric yarns. The elastomeric yarns move freely in the longitudinal direction in the bandage 14. The long-stretch elastomeric yarns can be spandex, preferably comprising about a 420 denier spandex. The long-stretch elastomeric yarns, and thus the elastic compression bandage 14, has a low rigidity index, or a low resistance to stretching, such that tension does not rise as rapidly when stretched as compared to “short-stretch” fabrics.

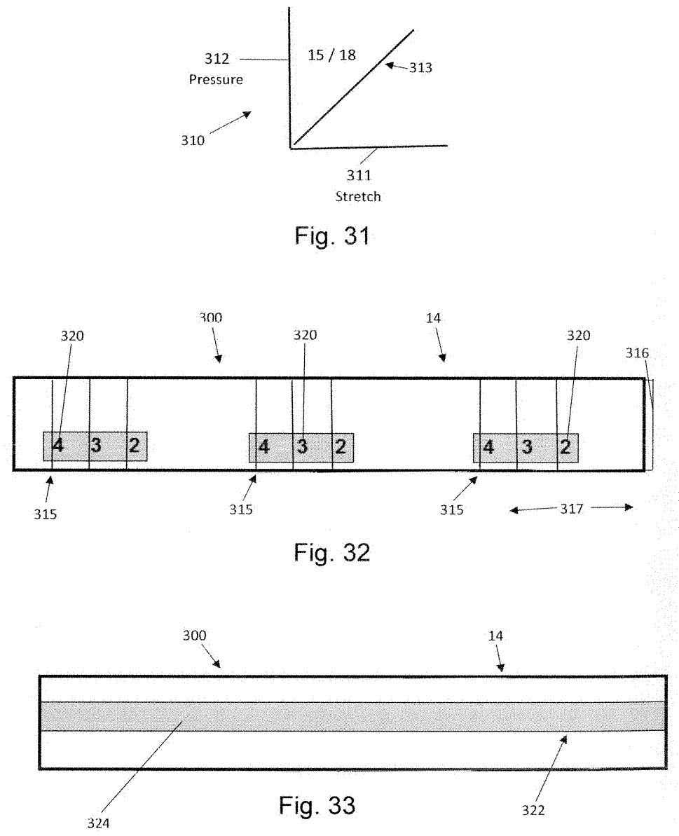

The long-stretch elastomeric yarns allow the elastic compression bandage 14 to comprise an approximate 1:1 stretch characteristic. That is, the change in compressive pressure provided by the bandage 14 is approximately proportional to the change in stretch of the bandage 14. A stress-strain curve 310 is a graphic representation of change in strain of a material with change in applied stress. As shown in FIG. 31, in a graph of stretch (stress) 311 on the x-axis and pressure (strain) 312 on the y-axis, the stress-strain curve 310 for embodiments of the elastic compression bandage 14 having a 1:1 stretch characteristic is an approximate linear relationship 313 (a 45-degree angle on the graph).

This 1:1 stretch characteristic of long-stretch elastomeric yarns in the compression bandage 14 overcomes the inability of short-stretch fibers in many commercial products to maintain pressures within an acceptable range when there is even a slight change in circumference of an underlying body part. For example, in some commercial compression bandages having short-stretch fibers, a small change in underlying circumference resulting in an increase in stretch (or tension) of as little as one percent, or length of 1/32 inch, causes a 10 mm Hg increase in pressure. That is, in a short-stretch yarn bandage, an increase from a 99% stretch providing 30 mm Hg pressure to a 100% stretch would cause 40 mm Hg pressure, and another one-percent increase to a 101% stretch would cause 50 mm Hg pressure. Therefore, short-stretch yarn bandages have difficulty maintaining desired pressures, or even safe pressures with small changes in underlying circumference. (“Pressures” herein are understood to be “interface pressures,” interpreted as the average force that is applied to the surface of a limb by a compressive device.)

Due to the 1:1 stretch characteristic, in embodiments of the long-stretch elastomeric bandages 14, changes in underlying circumference result in proportionate changes in pressure. As a result, the amount of compression can be calculated and controlled. This stretch characteristic of embodiments of the tension adjusting and stabilization system 300 and method allows compression to be maintained within a wide range of changes in circumference of underlying body parts. The long-stretch elastomeric bandage 14 provides a much larger range of stretch to cause the same change in pressure as in a small range of stretch in a short-stretch bandage. For example, each 10% increase in stretch of a long-stretch elastomeric bandage may produce an increase of 10 mm Hg pressure. To illustrate, an increase from an 90% stretch providing 30 mm IIg pressure to a 100% stretch would cause 40 mm Hg pressure, and an increase from a 100% stretch providing 40 mm Hg pressure to a 110% stretch would cause 50 mm Hg pressure.

Maintaining compression, or “interface pressure,” within a wide range of changes in circumference of an underlying body part can be explained by LaPlace's Law, as discussed herein. LaPlace's Law for cylindrically-shaped objects is expressed as P=T/r, where P is the internal pressure of the limb, T represents the wall tension across a slice of a cylindrical portion of a compression bandage, and r is the radius of the limb (the limb is approximated as a cylinder). LaPlace's Law implies that the pressure supplied by a compression bandage varies inversely with the radius of the limb. In other words, if tension is equal throughout the bandage 14, less pressure will be provided at a larger radius portion of the limb 20, such as the calf 26, than at a smaller radius portion of the limb 20, such as the ankle 25. For example, if tension (T) is the same throughout the wrapped bandage 14 (a constant 1), and the bandage 14 is wrapped to a pressure (P) of 40 mm Hg at the ankle 25 having a radius (r) of 2 inches, then at the calf 26 having a radius (r) of 4 inches, the pressure (P) would be 20 mm Hg:

| | Pressure = | Tension/ | radius |
|---|---|---|---|
| Ankle | 40 mmHg | 1 | 2 inches |
| Calf | 20 mmHg | 1 | 4 inches |

If the radius (r) of the limb 20 increases due to edema, tension (T) of the bandage 14 increases a known amount. As tension (T) increases, pressure (P) also increases. Likewise, when edema subsides, the radius (r) of the limb 20 decreases and tension (T) of the bandage 14 decreases a known amount. As tension (T) decreases, pressure (P) also decreases.

In addition, this stretch characteristic allows embodiments of the elastic compression bandage 14 to be applied having various amounts of tension so as to provide ranges of compressive pressure, for example, between about 10 mm Hg and 100 mm Hg. To produce a particular pressure with a certain stretch in a compression bandage having such a given (1:1) stretch characteristic, a known pressure can be set at the beginning of the stress-strain curve 310. Thus, tension on the bandage 14 can be changed at various levels on the body to provide predictable and maintainable pressures.

In addition, this 1:1 stretch characteristic of long-stretch elastomeric yarns in the compression bandage 14 allows hysteresis loss between stretch and recovery to be minimized. “Hysteresis” can be defined as the difference in the value of a material property according to its previous history, for example, the mechanical work lost when a textile is loaded and then relaxed. Given the approximate linear relationship 313 between change in pressure 312 and stretch 311 in the long-stretch elastomeric yarns, as underlying circumference increases, the compression bandage 14 stretches and provides a gradual increase in compression. Likewise, as underlying circumference decreases, the compression bandage 14 relaxes and provides a gradual decrease in compression. That is, the 1:1 stretch characteristic results in a continuity of change in pressure in response to changes in underlying circumference. A continuity of change in pressure allows graduated compression to be maintained, whether circumference is increasing or decreasing. Such a continuity, or gradualness, of change in pressure is reflected in the hysteresis curve, or loops, tending to the linear shape of the stress-strain curve 310.

Embodiments of the elastic compression bandage 14 of the tension adjusting and stabilization system and method can comprise woven or knitted construction, and can have various thicknesses.

Over time, there is a decrease in the amount of pressure supplied by compression fabric. This decrease in pressure can be partially attributed to a reduction in the size of a wearer's limb as swelling is reduced, as well as the compression system conforming to the shape of the body. However, a pressure reduction can also occur as a result of material fatigue. The latter broadly describes a loss in applied pressure resulting from a breaking down of the elastic elements in the compression fabric. In this case, the elastomer (such as spandex) relaxes within the fabric structure, and the fabric loses the ability to maintain the desired compressive force. This results in a loss of pressure, which reduces the therapeutic value of the compression fabric.

One form of material fatigue in a compression fabric is prolonged wear, which can be characterized as “creep.” “Creep” describes the tendency of all elastic materials to move slowly or deform permanently under prolonged exposure to a continuously applied mechanical load. “Creep” is defined as gradual stretching of a material under a constant load. In compression fabrics, creep has the detrimental effect of causing a loss of compression (pressure) over time. Different compression fabric structures exhibit different amounts of creep and thus different effects on interface pressure over time.

When a short-stretch, high rigidity index compression wrap is applied to provide 40 mm Hg pressure, due to the effects of creep, within a five-hour time period, the actual applied pressure reduces to about 25 mm Hg. When embodiments of the long-stretch, low rigidity index compression bandage 14 herein is applied to provide 40 mm Hg pressure, in a five-hour time period, the actual applied pressure is maintained at 38 mm Hg pressure, or within about five percent of the initial 40 mmHg pressure. That is, in embodiments of the tension adjusting and stabilization system 300 and method comprising the long-stretch compression bandage 14 herein, the detrimental effects of creep are avoided and compressive pressure is maintained within an acceptable range over time.

In another aspect, the tension adjusting and stabilization system 300 and method can comprise a tension/compression indication system in which a particular amount of stretch of the elastic compression bandage 14 reveals a unique indicator, such as a particular shape or design, representative of a particular amount of compressive pressure. The indicator can comprise one or more indicia knitted into, or printed onto, the bandage, or wrap, 14. Embodiments of such a tension/compression indication system are similar to those described with reference to FIGS. 4 and 5. In combination with the 1:1 stretch characteristic of the elastic compression bandage 14, embodiments of the tension/compression indication system provide for accurate adjustment and stabilization of the bandage/wrap 14.

In such an embodiment, the elastic compression bandage 14 of the tension adjusting and stabilization system 300 and method can comprise repeating series of lines 315 oriented transversely across at least a part of the width 316 of the bandage 14 at predetermined spaced-apart intervals along the length 317 of the bandage 14. The repeating series of lines 315 can be labeled with an indicator 320 representative of compressive pressure provided when the bandage 14 is stretched while wrapping about a body part such that lines 315 having like pressure indicator labels 320 are aligned in a turn. For example, as shown in FIG. 32, each line in a repeating series of three lines 315 can be labeled “4,” “3,” and “2.” The bandage 14 can be wrapped about a body part, such as a leg 20 or arm, so that lines with matching numbers can be aligned and wrapped together in turns. The pressure indicator numbers 320 can represent particular pressures. For example, a line labeled “4” can represent 40 mm Hg compressive pressure provided by the wrapped bandage 14 when two lines each labeled “4” are aligned in a turn. Likewise, a line labeled “3” can represent 30 mm Hg compressive pressure provided by the wrapped bandage 14 when two lines each labeled “3” are aligned in a turn. And, a line labeled “2” can represent 20 mm Hg compressive pressure provided by the wrapped bandage 14 when two lines each labeled “2” are aligned in a turn.

To provide graduated compression on a leg, the lines labeled “4” for 40 mm Hg pressure can be aligned in a turn about the ankle 25. As the bandage 14 is wrapped up the leg 20, the lines labeled “3” for 30 mm Hg pressure can be aligned at an appropriate level on the leg 20, for example, about the calf 26. And as the bandage 14 is wrapped further up the leg 20, the lines labeled “2” for 20 mm Hg pressure can be aligned at an appropriate higher level on the leg 20, for example, about the lower thigh. In this manner, the elastic bandage 14 can be easily wrapped having the proper tension and overlap 48 to provide graduated compression.

In another aspect, some embodiments of the elastic compression bandage 14 of the tension adjusting and stabilization system 300 and method comprise an elastic material having one color in an unstretched condition that changes to a different color in a stretched condition. In such embodiments, the different, stretched color corresponds to a predetermined amount of stretch applied to the material. In certain embodiments, the predetermined amount of stretch applied to the material corresponds to a predetermined amount of compressive pressure. The stretched color can comprise a first stretched color corresponding to a first predetermined amount of stretch and a second stretched color corresponding to a second predetermined amount of stretch. The first amount of stretch and the second amount of stretch can each correspond to a different predetermined amount of compressive pressure. In some embodiments, the elastic compression bandage 14 can further comprise a graduated compressive pressure garment, in which the different predetermined amounts of compressive pressure are graduated across selected locations in the garment.

For example, in one embodiment, the elastic bandage 14 can further include a pressure color indicator 322 along the length 317 of the bandage 14, as shown in FIG. 33. For example, the pressure color indicator 322 can be a strip of yarns 324 having a different color than surrounding yarns knit into the fabric. As the bandage fabric is stretched in the longitudinal direction, the pressure color indicator 322 turns from a bright, or solid, shade to an increasingly less solid, or lighter, shade. In another embodiment, as the bandage 14 fabric is stretched in the longitudinal direction, the pressure color indicator 322 turns from one color to another color. In such an embodiment, to provide graduated compression on a leg, the lines labeled “4” for 40 mm Hg pressure can be aligned in a turn about the ankle 25. In this way, a certain, desired beginning pressure can be applied at the distal portion of the leg 20. As the bandage 14 is wrapped up the leg 20 in the proximal direction, the bandage 14 can be stretched so that the pressure color indicator 322 visually appears to become an increasingly less solid, or lighter, shade (or a different color) to indicate graduation of pressure.

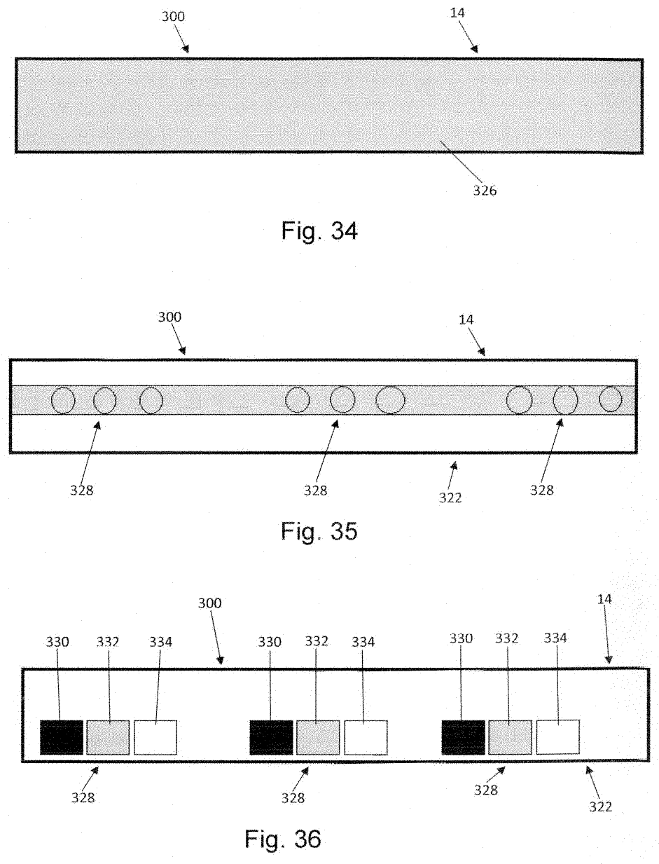

FIGS. 34-36 illustrate other embodiments of pressure color indicators 322 on the elastic bandage 14 useful to indicate that proper pressure is being applied at different levels. The indicator can be a color and/or pattern knitted in or printed on the wrap fabric. Different colors can be included such that when the same color is matched on the wrap when applied, a particular pressure is indicated.

In one such embodiment, as shown in FIG. 34, one side of the elastic bandage can comprise one color 326 and the other side of the bandage (not shown) can comprise a different color. The color 326 of one side can be matched as the bandage is being wrapped.

In another such embodiment, as shown in FIG. 35, the elastic bandage 14 can comprise the pressure color indicator 322 along the length 317 of the bandage 14 similar to that shown in FIG. 33. The longitudinal pressure color indicator 322 can further include a spaced-apart, repeating series of shapes 328, such as ovals, along its length. As the elastic bandage 14 is being wrapped, the ovals in a first one of the series can be matched to the ovals in a second series to provide a particular compressive pressure. By stretching the bandage 14 to have more tension and match the first series of ovals with a third series of ovals, the bandage 14 provides a greater known compressive pressure.

In another such embodiment, as shown in FIG. 36, the elastic bandage 14 can include the pressure color indicator 322 comprising a spaced-apart, repeating series of shapes 328, such as squares, along its length. Each of the first squares in each series can comprise a first color 330, each of the second squares in each series can comprise a second color 332, and each third square in each series can comprise a third color 334. As the elastic bandage 14 is being wrapped, the three colored squares in a first one of the series can be matched to the colored squares in another series to provide a particular compressive pressure.

In another aspect, embodiments of the tension adjusting and stabilization system 300 and method comprise a printed pattern on the elastic compression bandage 14 that allows accurate manipulation of tension. The pattern can be printed on the bandage in an unstretched condition 336. When the bandage 14 is placed under tension as it is being wrapped on a limb, the printed pattern changes to indicate a desired amount of tension has been reached and the amount of pressure being applied by that amount of tension. The disadvantage to conventional printed or knitted patterns is that those systems are qualitative only. For example, as one symbol or shape changes to another symbol or shape to indicate that the bandage has a certain amount of tension, there is no indication as to the particular amount of pressure being applied by that tension. Embodiments of the elastic compression bandage 14 herein provide a means for indicating both a qualitative degree of tension of the bandage as it is being wrapped on a limb and a quantitative amount of pressure being applied by that amount of tension.

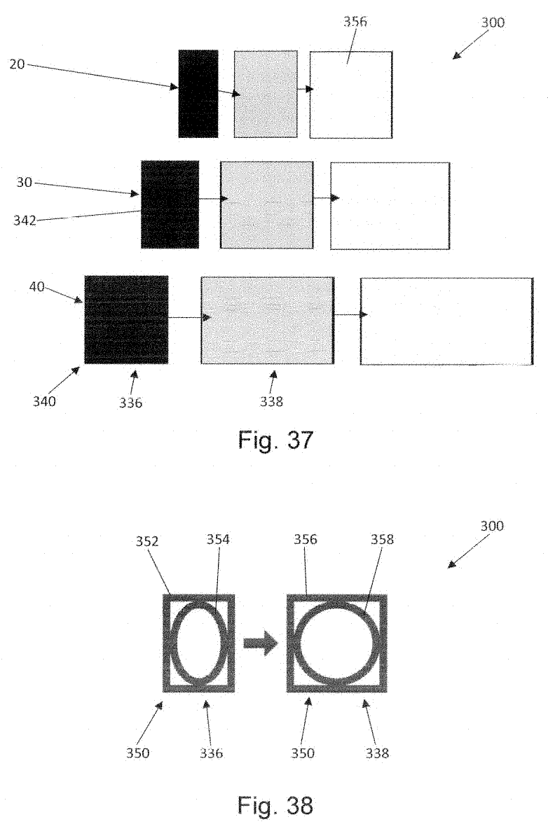

For example, as shown in FIG. 37, embodiments of the tension adjusting and stabilization system 300 and method can comprise the elastic compression bandage 14 having a repeating series of shape color tension and pressure indicators 340. The vertical row of dark (shown as black) rectangular boxes 342 can be printed onto the elastic compression bandage 14 in the unstretched condition 336 in a repeating series. The dark rectangular box 342 in the top row includes a marking such as “20” (to signify 20 mm Hg pressure) embedded but invisible within the box 342. Similarly, the dark rectangular boxes 342 in succeeding rows include increasingly larger numerical markings such as “30” in the middle row box 342 to signify 30 mm Hg pressure, and such as “40” in the bottom row dark box 342 to signify 40 mm Hg pressure.

As the compression bandage 14 is being wrapped about the limb 20, the dark rectangular boxes 342 stretch toward the shape of a square 356. When sufficient tension is applied on the bandage wrap 14 to achieve the pressure that the particular row indicates, the square shape 356 is reached in that row and the number signifying that pressure appears in the square 356 of that row. To adjust the amount of tension and pressure, the bandage 14 can be stretched further or relaxed to achieve the square shape 356 and corresponding pressure number in another row. For example, if the bandage 14 has been wrapped to achieve the square shape 356 and number “40” in the bottom row so as to provide a pressure of approximately 40 mm Hg, the tension on the bandage 14 can be relaxed so as to achieve the square shape 356 and number “30” in the middle row to provide a pressure of approximately 30 mm Hg; and the tension on the bandage 14 can be further relaxed so as to achieve a square shape and number 20 in the top row to provide a pressure of approximately 20 mm Hg. Accordingly, embodiments of the tension adjusting and stabilization system 300 and method comprising shape color tension and pressure indicators 340 provide the ability to manipulate tension and pressure.

In another example of an embodiment of the tension adjusting and stabilization system 300 and method, the elastic compression bandage 14 comprises the tension pressure indicator 350 printed in one or more locations on the bandage or wrap 14. As shown in FIG. 38, the tension pressure indicator 350 comprises a rectangle shape 352 having an oval shape 354 inside the rectangle shape 352 when the bandage is unstretched 336. When the bandage 14 is wrapped to a stretched position 338 such that the rectangle 352 becomes the square shape 356 and the oval 352 becomes a circle shape 358, the bandage 14 provides a predetermined amount of compressive pressure, for example, 20 mm Hg compressive pressure. Accordingly, the elastic compression bandage 14 herein provides a means for indicating both a qualitative degree of tension of the bandage 14 as it is being wrapped on the limb 20 and a quantitative amount of pressure being applied by that amount of tension.

In another embodiment, the elastic compression bandage 14 can further comprise a multi-layer wrap system, for example, two compression bandages 14, each having the tension pressure indicator 350 shown in FIG. 38 and providing 20 mm Hg compressive pressure when wrapped to the stretched position 338 such that the tension indicator 350 comprises the square shape 356 and the circle shape 358. A first of the two bandages 14 can be wrapped to the stretched position 338 such that the tension indicator 350 comprises the square shape 356 and the circle shape 358. A second of the two bandages 14 can be wrapped with a 50% overlap of the first bandage 14 such that the tension indicator 350 comprises the square shape 356 and the circle shape 358. Each bandage layer thereby provides 20 mm Hg pressure, and the two layers together provide a cumulative 40 mm Hg compressive pressure.

Various embodiments of the tension adjusting and stabilization system 300 and method can include combinations of features of the tension adjusting and stabilization system 300 and method, and elastic compression bandage, described herein. In particular, some embodiments of the elastic compression bandage 14 can include (1) long-stretch elastomeric yarns comprising an approximate 1:1 stretch characteristic so as to allow compression to be maintained within a wide range of changes in circumference of underlying body parts; (2) repeating series of lines 315 having pressure indicator numbers 320; (3) a pressure color indicator 322; (4) a shape color tension and pressure indicator 340; and/or (5) a tension pressure indicator 350. By controlling both the characteristics of the bandage 14 and the application of the wrap 14 (including tension and overlap), accurate interface pressures can be applied initially and maintained over time for therapeutic effects.

Although the subject matter described herein has been described with reference to particular embodiments, it should be recognized that these embodiments are merely illustrative of the principles of the subject matter described herein. Those of ordinary skill in the art will appreciate that a compression tension adjusting, stabilization, and sensing system and/or method of the subject matter described herein may be constructed and implemented in other ways and embodiments. Accordingly, the description herein should not be read as limiting the subject matter described herein, as other embodiments also fall within the scope of the subject matter described herein,

What is claimed is:

1. A tension adjusting and stabilization system, comprising:
an elastic compression bandage comprising long-stretch elastomeric yarns and an approximate 1:1 stretch characteristic,
wherein a change in length-wise stretch of the bandage results in an approximately proportional change in compressive pressure provided by the bandage.

2. The tension adjusting and stabilization system of claim 1, wherein each 10% increase in length-wise stretch of the bandage produces an increase of 10 mm Hg compressive pressure provided by the bandage.

3. The tension adjusting and stabilization system of claim 1, wherein the elastic compression bandage maintains pressure within about five percent of the initial applied pressure over at least a five-hour time period.

4. The tension adjusting and stabilization system of claim 1, wherein the elastomeric yarns comprise an about 420 denier spandex yarn.

5. The tension adjusting and stabilization system of claim 1, further comprising a repeating series of lines oriented transversely across at least a part of a width of the bandage at predetermined, spaced-apart intervals along a length of the bandage, each line within the repeating series of lines labeled with an indicator representative of a particular amount of compressive pressure such that when the bandage is stretched about a limb and the lines having matching pressure indicators are aligned, the amount of compressive pressure indicated by the matching pressure indicators is provided.

6. The tension adjusting and stabilization system of claim 1,
wherein the elastic compression bandage further comprises a pressure color indicator along a length of the bandage comprising one color in an unstretched condition that changes to a different color in a stretched condition,
wherein the different, stretched color corresponds to a predetermined amount of stretch applied to the bandage, and
wherein the predetermined amount of stretch applied to the bandage corresponds to a predetermined amount of compressive pressure.

7. The tension adjusting and stabilization system of claim 6, the pressure color indicator further comprising a spaced-apart, repeating series of shapes along the length of the bandage, wherein as the bandage is stretched, the shapes in a first one of the series are matched to the shapes in a second one of the series to provide a particular, known compressive pressure.

8. The tension adjusting and stabilization system of claim 7,
wherein a first shape in each series of shapes comprises a first color, a second shape in each series comprises a second color, and a third shape in each series comprises a third color,
wherein as the bandage is stretched, the three colored shapes in a first one of the series are matched to the colored shapes in another one of the series to provide a particular, known compressive pressure.

9. The tension adjusting and stabilization system of claim 1, further comprising a means for indicating a qualitative degree of tension of the bandage as it is being wrapped on a limb and a means for indicating a quantitative amount of pressure being applied by that degree of tension.

10. The tension adjusting and stabilization system of claim 9,
wherein the means for indicating a qualitative degree of tension further comprises a vertical row of dark rectangular boxes printed onto the elastic compression bandage in an unstretched condition,
wherein the means for indicating a quantitative amount of pressure further comprises a mark representing a unique amount of pressure embedded in each of the dark rectangular boxes invisible in the unstretched condition, and
wherein when sufficient tension is applied on the bandage to achieve the pressure invisible within one of the rectangular boxes, that rectangular box becomes a square shape and the unique amount of compressive pressure embedded in that rectangular box becomes visible.

11. The tension adjusting and stabilization system of claim 1,
the elastic compression bandage further comprising a tension pressure indicator printed in one or more locations on the bandage and comprising a rectangle shape having an oval shape inside the rectangle shape when the bandage is unstretched,
wherein when the bandage is wrapped to a stretched position such that the rectangle shape becomes a square shape and the oval shape becomes a circle shape, the bandage provides a predetermined amount of compressive pressure.

12. A tension adjusting and stabilization method, comprising:
providing an elastic compression bandage comprising long-stretch elastomeric yarns and an approximate 1:1 stretch characteristic, wherein a change in length-wise stretch of the bandage results in an approximately proportional change in compressive pressure provided by the bandage; and
wrapping the bandage with a particular amount of tension at a certain level on a limb, thereby providing a known amount of pressure at that level of the limb.

13. The tension adjusting and stabilization method of claim 12,
providing two of the elastic compression bandages, each further comprising a tension pressure indicator printed in one or more locations on the bandage and comprising a rectangle shape having an oval shape inside the rectangle shape when the bandage is unstretched, wherein when the bandage is wrapped to a stretched position such that the rectangle shape becomes a square shape and the oval shape becomes a circle shape, the bandage provides a predetermined amount of compressive pressure;
wrapping a first of the two bandages to the stretched position such that the tension pressure indicator comprises the square and the circle; and
wrapping a second of the two bandages with a 50% overlap of the first bandage such that the tension pressure indicator comprises the square and the circle,
wherein each bandage provides the same amount of pressure, and the two bandages together provide a cumulative pressure approximately twice that of one bandage.

14. A tension adjusting and stabilization system, comprising:
a wearable compressive pressure device comprising
an elastic compression bandage comprising long-stretch elastomeric yarns and an approximate 1:1 stretch characteristic, wherein a change in length-wise stretch of the bandage results in an approximately proportional change in compressive pressure provided by the bandage;
an electrically conductive yarn
(a) comprising a 70 denier conductive yarn having 24-68 filaments,
(b) knit vertically in a selected number of adjacent wales along a longitudinal axis of the device,
(c) knit tightly such that stitch loops in adjacent courses along the wales are compacted together to enhance yarn and fiber contact, and
(d) comprising a transmission circuit configured to transmit an electrical signal representing a compressive pressure value in an area of a body to a connection point on the transmission circuit;
a sensor connectable to the transmission circuit and configured to sense compressive pressure in the area of a body to which the device is applied; and
a data processor or display unit connectable to the transmission circuit and configured to display the transmitted compressive pressure value.
